(19) United States
(12) Patent Application Publication
(10) Pub. No.: US 2021/0401566 A1
(43) Pub. Date: Dec. 30, 2021

(54) STENT GRAFT SYSTEMS AND METHODS WITH CUFF AND LIMB

(71) Applicant: Endologix LLC, Irvine, CA (US)

(72) Inventors: Mark Geusen, Irvine, CA (US); Teri Woodson, Irvine, CA (US); Christopher Staudenmayer, Irvine, CA (US); Dennis Parson, Irvine, CA (US); Dion Thurow, Irvine, CA (US); Ryan Goff, Irvine, CA (US); Kaushik Patel, Irvine, CA (US); Aric Stone, Irvine, CA (US); Dale Ehnes, Irvine, CA (US); Craig Welk, Irvine, CA (US)

(21) Appl. No.: 17/279,000

(22) PCT Filed: Sep. 23, 2019
(86) PCT No.: PCT/US2019/052459

§ 371 Date: Mar. 23, 2021

Related U.S. Application Data
(60) Provisional application No. 62/735,771, filed on Sep. 24, 2018.

(51) Int. Cl. A61F 2/07

(52) U.S. Cl. CPC A61F 2/07; A61F 2002/067

(57) ABSTRACT

A stent graft system includes a first graft, a second graft, and a third graft. Each of the first graft, the second graft, and the third graft forms a single lumen. When deployed, the first graft, the second graft, and the third graft are coupled together within an aorta.

### CROSS-REFERENCE TO RELATED PATENT APPLICATIONS

This application claims priority from U.S. Provisional Patent App. Ser. No. 62/735,771, filed Sep. 24, 2018, the entire contents of which are incorporated by reference herein.

### FIELD

The present technology relates generally to endoluminal vascular prostheses and methods of placing/deploying such prostheses. More particularly, various arrangements disclosed herein relate to stent graft systems and to methods of placing/deploying such stent graft systems for treating aortic aneurysms.

### BACKGROUND

Aneurysms are enlargements or bulges in blood vessels that are often prone to rupture and which therefore present a serious risk to a patient. Aneurysms may occur in any blood vessel but are of particular concern when they occur in the cerebral vasculature or the aorta.

Abdominal aortic aneurysms (AAAs) are classified based on their locations within the aorta as well as their shapes and complexity. Aneurysms that are found below the renal arteries are referred to as infrarenal abdominal aortic aneurysms. Suprarenal abdominal aortic aneurysms occur above the renal arteries. Thoracic aortic aneurysms (TAAs) occur in the ascending, transverse, or descending part of the upper aorta. Infrarenal aneurysms are the most common, representing about 70% of all aortic aneurysms. Suprarenal aneurysms are less common, representing about 20% of the aortic aneurysms. TAAs are the least common and often the most difficult to treat.

The most common form of aneurysm is “fusiform,” where the enlargement extends about the entire aortic circumference. Less commonly, the aneurysms may be characterized by a bulge on one side of the blood vessel attached at a narrow neck. TAAs are often dissecting aneurysms caused by hemorrhagic separation in the aortic wall, usually within the medial layer. A common treatment for each of these types and forms of aneurysm is open surgical repair. Open surgical repair is quite successful in patients who are otherwise reasonably healthy and free from significant co-morbidities. Such open surgical procedures are problematic, however, because access to the abdominal and thoracic aortas is difficult to obtain, and because the aorta must be clamped off, placing significant strain on the patient's heart.

Endoluminal grafts have come into widespread use for the treatment of aortic aneurysms in patients. A typical endograft procedure utilizes a stent graft placement to treat the aneurysm. The purpose of the graft is generally to isolate the diseased portion of the aortic wall from the aortic blood pressure and prevent further dilatation or rupture of the diseased portion of the aortic wall. In general, endoluminal repairs access the aneurysm “endoluminally” through either or both iliac arteries. The grafts are then implanted. Successful endoluminal procedures have a much shorter recovery period than open surgical procedures.

### SUMMARY OF THE DISCLOSURE

Various stent graft systems and methods described herein are directed to treating aneurysms. In some arrangements, a stent graft system includes a first graft, a second graft, and a third graft. Each of the first graft, the second graft, and the third graft forms a single lumen. When deployed, the first graft, the second graft, and the third graft are coupled together within an aorta.

In some arrangements, the second graft and the third graft are inserted into the single lumen of the first graft when deployed. In some arrangements, a portion of the first graft is placed in a proximal neck region of the aorta when deployed. A portion of the second graft is placed in a first iliac artery of the aorta when deployed. A portion of the third graft is placed in a second iliac artery of the aorta when deployed.

In some arrangements, the first graft, the second graft, and the third graft are separate grafts before being deployed. In some arrangements, the stent graft system further includes an inflatable fill structure at least partially surrounding the first graft. The inflatable fill structure expands within the aorta when deployed. A seal component coupled to the first graft. The seal component forms a seal in a proximal neck region of the aorta.

In some arrangements, the seal component is filled to a higher pressure than a pressure of the inflatable fill structure. In some examples, the seal component and the inflatable fill structure are filled using different channels. In some examples, the inflatable fill structure, when deployed, at least partially surrounds proximal ends of the second graft and the third graft that are docked within the single lumen of the first graft. In some examples, the inflatable fill structure is coupled to the first graft. In some examples, the second graft and the third graft dock within the single lumen of the first graft in a docking zone. The inflatable fill structure, in an inflated state, surrounds at least portions of the second graft and the third graft that are outside of the docking zone. In some examples, the inflatable fill structure is coupled to the first graft. The inflatable fill structure, in an inflated state, surrounds portions of the second graft and the third graft that are inside of iliac arteries when deployed.

In some examples, the second graft and the third graft dock within the single lumen of the first graft in a docking zone. The first graft includes a support inflatable fill structure coupled to a portion of the first graft in the docking zone. The support inflatable fill structure is inflated to provide structural integrity to the first graft. In some examples, the support inflatable fill structure is inflated before or while the inflatable fill structure is inflated. In some examples, the second graft and the third graft dock within the single lumen of the first graft in a docking zone. The first graft includes a wire-wound stent component coupled to a portion of the first graft in the docking zone. The wire-wound stent component includes a plurality of wire-wound rings. In some examples, wherein the single lumen of the first graft is open at the wire-wound stent component. In some examples, the second graft and the third graft dock within the single lumen of the first graft in a docking zone. The first graft includes a wire-wound stent ring coupled to a portion of the first graft in the docking zone, the wire-wound stent ring includes a single ring of wire-wound stent.

In some examples, the inflatable fill structure is more compliant than the seal component. In some examples, the inflatable fill structure forms a funnel shape in an inflated state. In some examples, the inflatable fill structure forms the funnel shape by extending a portion of the inflatable fill structure adjacent to walls of the aorta along the walls of the aorta farther than another potion of the of the inflatable fill structure abutting and adjacent to the first graft. In some examples, the inflatable fill structure is a bifurcated inflatable fill structure that, when in the inflated state, forms two lumens for receiving the second graft and the third graft.

In some arrangements, the stent graft system further includes a first inflatable fill structure at least partially surrounding the first graft, a second inflatable fill structure at least partially surrounding the second graft, and a third inflatable fill structure at least partially surrounding the third graft, the first inflatable fill structure, the second inflatable fill structure, and the third inflatable fill structure are separate inflatable fill structures that expand within the aorta when deployed. In some examples, the first inflatable fill structure expands into the single lumen of the first graft.

In some examples, the second inflatable fill structure surrounds a portion but not all of an outer surface the second graft. The third inflatable fill structure surrounds a portion but not all of an outer surface the third graft. In some examples, the second inflatable fill structure surrounds an entirety of an outer surface the second graft. The third inflatable fill structure surrounds an entirety of an outer surface the third graft.

In some arrangements, the stent graft system further includes an inflatable fill structure coupled to the first graft. The inflatable fill structure, while in an inflated state, forms a seal in a proximal neck region of the aorta. The second graft and the third graft dock within the single lumen of the first graft in a docking zone. The inflatable fill structure, while in the inflated state, surrounds at least portions of the second graft and the third graft that are outside of the docking zone. The inflatable fill structure, while in the inflated state, at least partially surrounding the first graft.

In some arrangements, the stent graft system further includes a first inflatable fill structure at least partially surrounding the second graft and a second inflatable fill structure at least partially surrounding the third graft. The first inflatable fill structure and the second inflatable fill structure expand within the aorta and surrounds at least partially the first graft when deployed. In some examples, each of the second graft and the third graft includes a wire-wound stent component. The wire-wound stent component includes a plurality of wire-wound rings. In some examples, the first inflatable fill structure and the second inflatable fill structure are fixed to portions of the second graft and the third graft that are inserted into the lumen of the first graft. The first inflatable fill structure and the second inflatable fill structure expand within the lumen of the first graft. In some examples, the first inflatable fill structure and the second inflatable fill structure expand into the lumen of the first graft.

In some arrangements, the second graft and the third graft dock within the single lumen of the first graft in a docking zone. The first graft includes at least one support inflatable fill structure coupled to a portion of the first graft in the docking zone. The second graft and the third graft are inserted into an opening of each of the at least one support inflatable fill structure when the second graft and the third graft are inserted into the single lumen of the first graft in the docking zone. The at least one support inflatable fill structure provides a seal with respect to the first graft, the second graft, and the third graft within the lumen of the first graft. In some examples, the opening has a bi-lobe shape.

In some arrangements, the second graft and the third graft dock within the single lumen of the first graft in a docking zone. The first graft includes at least one internal support component coupled to a portion of the first graft in the docking zone. The internal support component expands within the single lumen of the first graft when inflated and forms a seal around the second graft and the third graft when the second graft and the third graft are inserted into the single lumen of the first graft in the docking zone. In some arrangements, the first graft includes a seal component coupled to a distal end of the first graft.

In some arrangements, the second graft and the third graft dock within the single lumen of the first graft in a docking zone. The first graft includes an internal inflatable fill structure coupled to the first graft in the docking zone. The internal inflatable fill structure expands within the single lumen of the first graft when inflated and forms a seal around the second graft and the third graft when the second graft and the third graft are inserted into the single lumen of the first graft in the docking zone. The internal inflatable fill structure forms a bifurcated lumen.

In some examples, the bifurcated lumen is formed by inflating a proximal portion of the internal inflatable fill structure around a first balloon having a circular or oval cross section and a distal portion of the internal inflatable fill structure around a second balloon having a bi-lobe cross section.

In some arrangements, the first graft includes a laminated stent component. In some examples, the laminated stent component includes Teflon-laminated nickel-titanium (NiTi)-stents.

In some arrangements, the stent graft system includes an anchor configured to attach the first graft to the aorta. The anchor includes hooks or barbs. In some arrangements, the anchor is located on a stent ring of the first graft. In some arrangements, the first graft includes a support structure coupled to the first graft, the support structure being in the lumen of the first graft. In some example, the support structure includes helix-shaped polymer support rings.

In some arrangements, the stent graft system includes a graft forming a lumen and at least one support component embedded in the graft. Each of the at least one support component is a polymer ring surrounding the graft. At least a portion of each of the at least one support component is coupled to an external surface of the graft, the external surface faces away from the lumen. In some examples, the at least one support component includes a first support component and a second support component. The first support component is located on a first end of the graft. The second support component is located on a second end of the graft. In some examples, the at least one support component includes three or more support components spaced apart from each other along the graft. In some examples, the graft further forms a bifurcated feature including two additional lumens that receive limb stent grafts. In some examples, the graft further includes inner sleeves or rings in the lumen that receive limb stent grafts.

In some arrangements, a system includes a proximal extension inflatable fill structure, the proximal extension inflatable fill structure forms a seal in a proximal neck region of an aorta when the proximal extension inflatable fill structure is inflated. The system further includes at least one lumen formed by the proximal extension inflatable fill structure when the proximal extension inflatable fill structure is inflated. Each of the at least one lumen receives a limb stent graft, the at least one lumen being located in the proximal neck region when the proximal extension inflatable fill structure forms the seal in the proximal neck region. In some arrangements, the system further includes an anchor coupled to the proximal extension inflatable fill structure. In some examples, a length of the anchor is 30 mm. In some examples, a width of the proximal extension inflatable fill structure when filled is 20 mm. In some examples, the proximal extension inflatable fill structure is an endobag.

### BRIEF DESCRIPTION OF THE DRAWINGS

FIG. 1 is a cross-sectional view of an example infrarenal aortic aneurysm of a patient.

FIG. 2 is a cross-sectional view of an example stent graft system deployed across an aneurysm according to various arrangements.

FIG. 3A is a cross-sectional view of an example stent graft system deployed across an aneurysm according to various arrangements. FIG. 3B is another cross-sectional view of the example stent graft system (FIG. 3A) deployed across an aneurysm according to various arrangements.

FIG. 4A is a cross-sectional view of an example stent graft system deployed across an aneurysm according to various arrangements. FIG. 4B is another cross-sectional view of the example stent graft system (FIG. 4A) deployed across an aneurysm according to various arrangements.

FIG. 5 is a cross-sectional view of an example stent graft system deployed across an aneurysm according to various arrangements.

FIG. 6A is a cross-sectional view of an example stent graft system deployed across an aneurysm according to various arrangements. FIG. 6B is another cross-sectional view of the example stent graft system (FIG. 6A) deployed across an aneurysm according to various arrangements.

FIG. 7 is a cross-sectional view of an example stent graft system deployed across an aneurysm according to various arrangements.

FIG. 8 is a cross-sectional view of an example stent graft system deployed across an aneurysm according to various arrangements.

FIG. 9 is a cross-sectional view of an example stent graft system deployed across an aneurysm according to various arrangements.

FIG. 10 is a cross-sectional view of an example stent graft system deployed across an aneurysm according to various arrangements.

FIG. 11A is a cross-sectional view of an example stent graft system deployed across an aneurysm according to various arrangements. FIG. 11B is another cross-sectional view of the example stent graft system (FIG. 11A) deployed across an aneurysm according to various arrangements.

FIG. 12 is a cross-sectional view of an example stent graft system deployed across an aneurysm according to various arrangements.

FIG. 13A is a cross-sectional view of an example stent graft system deployed across an aneurysm according to various arrangements. FIG. 13B is another cross-sectional view of the example stent graft system (FIG. 13A) deployed across an aneurysm according to various arrangements.

FIG. 14 is a cross-sectional view of an example stent graft system deployed across an aneurysm according to various arrangements.

FIG. 15A is a cross-sectional view of an example stent graft system deployed across the aneurysm (FIG. 1) according to various arrangements. FIG. 15B is another cross-sectional view of the example stent graft system (FIG. 15A) deployed across the aneurysm (FIG. 1) according to various arrangements. FIG. 15C is yet another cross-sectional view of the example stent graft system (FIG. 15A) deployed across the aneurysm (FIG. 1) according to various arrangements.

FIG. 16 is a cross-sectional view of an example stent graft system deployed across an aneurysm according to various arrangements.

FIG. 17 is a cross-sectional view of an example stent graft system deployed across the aneurysm 14 (FIG. 1) according to various arrangements.

FIG. 18 is a cross-sectional view of an example stent graft system deployed across the aneurysm 14 (FIG. 1) according to various arrangements.

FIG. 19 is a cross-sectional view of an example stent graft system deployed across the aneurysm 14 (FIG. 1) according to various arrangements.

FIGS. 20A, 20B, 20C, 20D, 20E, 20F, 20G, 20H, 20I, 20J, 20K, and 20L illustrate examples of a proximal graft according to various arrangements.

FIGS. 21A, 21B, 21C, and 21D illustrate examples of a stent graft according to various arrangements.

FIG. 22A illustrates an example proximal extension inflatable fill structure of a stent graft system according to various arrangements.

FIG. 22B is a cross-sectional view of the stent graft system (FIG. 22A) deployed across the aneurysm 14 (FIG. 1) according to various arrangements.

FIG. 23 illustrate an example proximal extension inflatable structure of a stent graft system according to various arrangements.

### DETAILED DESCRIPTION

Various arrangements are described hereinafter. It should be noted that the specific arrangements are not intended as an exhaustive description or as a limitation to the broader aspects discussed herein. One aspect described in conjunction with a particular arrangement is not necessarily limited to that arrangement and may be practiced with any other arrangement(s).

Various arrangements disclosed herein relate to a stent graft system that includes a single-lumen proximal graft coupled to an inflatable fill structure (e.g., an endobag) and limb stent grafts (limbs) that may be coupled to one or more inflatable fill structures. Such a stent graft system includes one or more additional inflatable fill structures (e.g., those coupled to the limbs) for sac management. Sac management refers to the management of support in the aneurysm sac. An anchor (e.g., a component of the stent graft system used for fixing or attaching the stent graft system to the aorta) is separate from a seal component and separate from the sac management component (e.g., the inflatable fill structures), resulting in a more robust design as compared to the designs of other stent graft systems. In some implementations, the seal component coupled to the proximal graft is sized appropriately (e.g., by including a wide seal ring), which improves placement accuracy. In some implementations, the stent graft system includes a large single-lumen proximal graft (e.g., having a large bore diameter), which is easier to cannulate than other devices (e.g., stent graft systems having a graft component with a bifurcated lumen) and thus needs less procedure time and fluoro time as compared to the other devices. Various arrangements of the stent graft systems are cheaper to manufacture as compared to other devices (e.g., the devices having a graft component with a bifurcated lumen) because the single-lumen proximal graft is cheaper to manufacture than a graft with a bifurcated lumen.

Various arrangements disclosed herein relate to a stent graft system that includes a proximal graft having a proximal suprarenal self-expanding stent with fixation features coupled to a dual-lumen polymer filled inflatable fill structure (e.g., a dual-lumen polymer filled endobag). As compared to current AAA devices, the disclosed stent graft systems include a custom neck sealing and proximal fixation to the sac management feature of stent graft systems. For example, the disclosed stent graft systems separate proximal fixation, neck seal, cuff-to-stent-graft seal, and stent-graft-to-sac seal. Furthermore, the seal component (e.g., the cuff) of such a stent graft system is sized appropriately by having a wide sealing area below a fixation feature (e.g., the fixation stent frame), thus improving placement accuracy. In some implementations, a separate neck seal (e.g., a custom neck seal) can produce higher sealing pressures than the sealing pressures of seals of other stent graft systems, which allows the neck seal to last longer.

In some cases, the more design requirements or functionalities are placed on a design feature (e.g., a discrete, separate component) of a stent graft system, the less efficient the design feature can become. Various arrangements of the stent graft system as described herein include separate design features or components for fixation, sealing, and sac management.

Some arrangements of the stent graft system include a proximal graft that is single-lumen, referred to herein as a single-lumen proximal graft. The single-lumen proximal graft has a bore diameter and an overall length similar to a diameter and overall length of an aortic body. A single-lumen proximal graft is less complex as a structure/component and is easier to manufacture than a bifurcated lumen. An unsupported portion of the single-lumen proximal graft has a sufficient length (e.g., approximately 30 mm) that is above limb edges (that are inside the single-lumen proximal graft) for bailout procedures (e.g., deploying a Palmaz stent inside single-lumen proximal graft) or to build up from the stent graft system to treat complex AAAs and TAAs.

In some arrangements, the proximal graft includes a suprarenal laser-cut stent with coils attached thereon. In some examples, the suprarenal stent has a stent shorter than that of some current stent graft systems to eliminate free crowns. A shorter stent allows for a larger neck angle indication due to an improved stent graft flexibility. As such, the suprarenal stent in the stent graft systems described herein is shorter and has fewer crowns and fewer anchor, which allows the stent graft systems to be used for smaller treatment sizes. That is, the stent graft systems described herein is a low-profile delivery system used for small treatment sizes.

In some arrangements, the seal component includes a polytetrafluoroethylene (PTFE) polymer seal ring that is wider as compared to the seal rings on other devices. A wider seal ring improves placement accuracy given that even if the stent graft system is placed lower (e.g., 1 mm lower) than an optimal position, the wider seal ring can nevertheless provide a tight seal in a neck of an aorta. Furthermore, the wider seal ring has a wider treatment diameter range, which means fewer number of seal ring sizes (and fewer number of stock keeping unit (SKUs)) are needed for treating the entire vessel treatment range. In some arrangements, a neck length of the aortic neck region in which the seal component is configured to be deployed can be shorter than the neck lengths in which the seal components of other devices are configured to be deployed. Furthermore, the wide seal ring can improve the neck angle indication.

In some arrangements, the proximal graft includes an inflatable fill structure (e.g., an endobag) attached thereto. For sac management, the inflatable fill structure is deployed at a location below (in the distal direction of) the seal component. The inflatable fill structure can include a dedicated fill port through which the inflatable fill structure is filled or inflated, in some examples. In other examples, the inflatable fill structure and the seal component are filled using a same fill port, thus reducing the delivery system profile.

In some examples, the inflatable fill structure can be made from PTFE or a low-durometer polyurethane. In some cases, PTFE is used for the inflatable fill structure given that PTFE can be thermally bonded to the PTFE bore of the proximal graft and/or the PTFE bore of the seal component. In some examples in which the inflatable fill structure is made from PTFE, a larger inflatable fill structure is implemented given that PTFE is less elastic, where such a large inflatable fill structure can increase the device profile. On the other hand, in some examples in which the inflatable fill structure is made from polyurethane, less material is needed for the inflatable fill structure than the materials needed for an inflatable fill structure made from PTFE given that polyurethane is more elastic than PTFE, where less material can reduce the device profile. However, polyurethane cannot be thermally bonded to the PTFE proximal graft and/or the PTFE seal component easily. Thus, if polyurethane is used for the inflatable fill structure, the polyurethane of the inflatable fill structure is sutured to the PTFE proximal graft and/or the PTFE seal component. In some cases, blood can enter a space between the bore of the proximal graft and an inner lumen of the inflatable fill structure, thus pressurizing the inner lumen.

With respect to docking, a distal proximal graft section in which limbs dock has a universal bore size for all proximal graft sizes, such that the proximal graft can taper in or out to a desired vessel size. In various arrangements, the proximal graft is supported by a wire-wound stent to avoid kinking the proximal graft lumen in angulated anatomy. In some arrangements, a distal proximal graft universal docking section is supported by a wire-wound stent for docking the limbs with sufficient radial force to minimize the likelihood of dislodgement between the proximal graft and the limbs, so as to minimize Type III Endoleaks. With respect to an unsupported proximal graft, another inflatable fill structure (e.g., a balloon placed inside of the proximal graft) can be used as the inflatable fill structure (e.g., the endobag) is being filled to avoid proximal graft collapsing. In some examples, the balloon can be integrated with the proximal graft delivery system. That is, the balloon can be filled using the catheter used to fill the proximal graft. Alternatively, the balloon can be filled using a catheter separate from the catheter used for the proximal graft delivery system.

In some arrangements, limbs described herein can be self-expanding PTFE-covered stents or balloon-expandable PTFE-covered stents. The self-expanding PTFE-covered stents have sufficient radial structural integrity (e.g., radial force) to prevent lumen collapse during as the inflatable fill structure (e.g., the endobag) is being filled. On the other hand, the balloon-expandable PTFE-covered stents need a balloon to expand the stent. As such, the self-expanding PTFE-covered stents have a smaller device profile as compared to the device profile of the balloon-expandable PTFE-covered stents.

With respect to fixation (e.g., docking, deployment, insertion, and so on) in which the proximal graft is coupled to the limbs, in some arrangements, the limb diameters of at least one limb docked or to be docked in a docking zone (or an overlap zone) is smaller than a diameter of the bore of the proximal graft. In such examples, the inflatable structure (e.g., the endobag) around each limb inside the docking zone can seal off gutters that are typically present when docking the at least one limb inside a larger bore. In alternative arrangements, the sum of limb diameters of the at least one limb docked or to be docked in the docking zone is greater than the diameter of the proximal graft bore. In the example in which two limbs are docked into the docking zone, the cross sections of the two limbs are compressed into D-shapes inside the proximal graft bore, creating joint separation resistance due to radial force exerted by the limbs against the proximal graft bore. In such arrangements, the limbs can each include an inflatable fill structure (e.g., an endobag) in the docking zone to seal off any remaining gutters.

With respect to sac management, in various arrangements, the limbs have inflatable fill structures (e.g., endobags) attached to PTFE-covered stents of the limbs. The inflatable fill structure can cover an entire length of a limb, including part of the limb that is in the docking zone of the proximal graft. The inflatable fill structure can seal off the aneurysm sac and create a seal in the distal iliacs. In some arrangements, the inflatable fill structures for the proximal graft and/or the limbs may be optional, depending on whether a type II Endoleak is present.

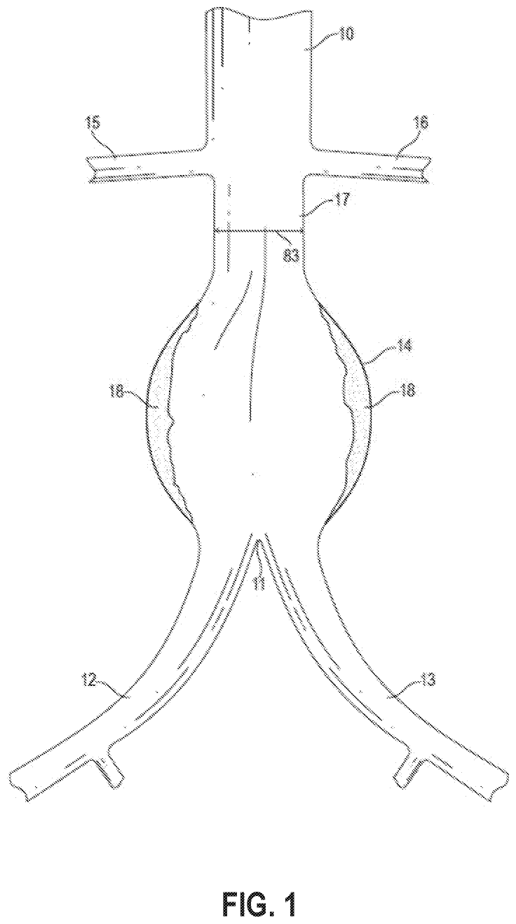

FIG. 1 is a cross-sectional view of an example infrarenal aortic aneurysm 14 of a patient. Referring to FIG. 1, an aorta 10 branches at an aortic bifurcation 11 into two iliac arteries 12 and 13. A sac of the aneurysm 14 corresponds to a bulged section of the aorta 10. The infrarenal aortic aneurysm 14 is located below (in a distal direction relative to) renal arteries 15 and 16. A segment of the aorta 10 between the renal arteries 15 and 16 and the sac of the aneurysm 14 is referred to as a proximal neck region 17. The proximal neck region 17 has a diameter 83 that is different for different patients. Often, a mural thrombus 18 forms on an inside wall of the sac of the aneurysm 14. The mural thrombus 18 may be omitted in other Figures for clarity.

With reference to FIG. 1, the dimensions of the aneurysm 14 can vary greatly from patient to patient. The diameter of the proximal neck region 17 may vary, for example, from 18 mm to 34 mm. The distance from the aortic bifurcation 11 to the renal arteries 15 and 16 may vary, for example, from 80 mm to 160 mm. The diameters of the right and left iliac arteries 12 and 13 may not be the same. The diameters of the iliac arteries 12 and 13 at the aortic bifurcation 11 may vary, for example, from 8 mm to 20 mm. One or both of the iliac arteries 12 and 13 may be aneurysmal with greatly enlarged diameters, for example, of more than 30 mm.

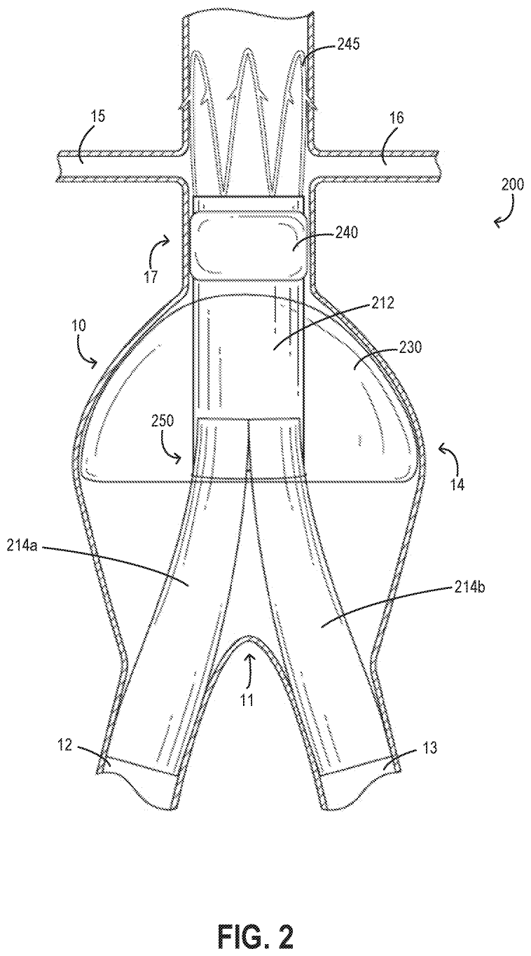

FIG. 2 is a cross-sectional view of an example stent graft system 200 deployed across the aneurysm 14 (FIG. 1), according to various arrangements. Referring to FIGS. 1 and 2, the stent graft system 200 is an endovascular graft system, an infrarenal prosthesis, and so on. The stent graft system 200 includes a proximal graft 212, a first limb stent graft 214a, a second limb stent graft 214b, an inflatable fill structure 230, a seal component 240, and an anchor 245.

In some arrangements, the proximal graft 212 can be a graft component made from a graft material without stents in some examples. The proximal graft 212 has a proximal end, a distal end, and an external surface. The proximal end of the proximal graft 212 is the end of the proximal graft 212 that is closer to or in the proximal neck region 17 when deployed. As shown, the proximal end of the proximal graft 212 can be placed in the proximal neck region 17 when deployed. The distal end of the proximal graft 212 is the end of the proximal graft 212 that is closer to the aortic bifurcation 11 when deployed. As shown, the distal end of the proximal graft 212 can be placed into the sac of the aneurysm 14, which is between the proximal neck region 17 and the aortic bifurcation 11. The external surface of the proximal graft 212 faces walls/surfaces of the aorta 10 and faces away from a tubular lumen of the proximal graft 212.

In some arrangements, the limb stent grafts 214a and 214b can be referred to as limbs. In some examples, each of the limb stent grafts described herein (e.g., the limb stent grafts 214a and 214b) includes graft material with stents. In other examples, a limb stent graft may be simply graft material without stents. Each of the limb stent grafts 214a and 214b can be a self-expanding PTFE-covered stent or a balloon-expandable PTFE-covered stent. Each of the first limb stent graft 214a and the second limb stent graft 214b has a proximal end, a distal end, and an external surface. The proximal end of each of the limb stent grafts 214a and 214b is an end of each of the limb stent grafts 214a and 214b that is closer to the proximal neck region 17 when deployed. As shown, the proximal ends of the limb stent grafts 214a and 214b can be placed in the sac of the aneurysm 14. The distal end of each of the limb stent grafts 214a and 214b is an end of each of the limb stent grafts 214a and 214b that is closer to or in the iliac arteries 12 and 13. As shown, the distal end of the first limb stent graft 214a can be placed in the iliac artery 12 when deployed, and the distal end of the second limb stent graft 214b can be placed in the iliac artery 13 when deployed. The limb stent grafts 214a and 214b can be placed at or adjacent to the aortic bifurcation 11. The external surface of each of the limb stent grafts 214a and 214b faces the walls/surfaces of the aorta 10 and faces away from a tubular lumen of each of the limb stent grafts 214a and 214b.

The stent graft system 200 can be deployed across the aneurysm 14 in any suitable manner. In one example, the distal ends of limb stent grafts 214a and 214b are first placed into the iliac arteries 12 and 13, respectively. The distal end of the proximal graft 212 is then placed over and around the proximal ends of the limb stent grafts 214a and 214b, such that the proximal ends of the limb stent grafts 214a and 214b are inserted into the tubular lumen of the distal end of the proximal graft 212. The portions of the limb stent grafts 214a and 214b that are inserted into the proximal graft 212 and the portion of the proximal graft 212 that surrounds the limb stent grafts 214a and 214b are in a docking zone 250 (an overlap zone or a distal proximal graft universal docking section in which the proximal graft 212 and the limb stent grafts 214a and 214b overlap). When the distal end of the proximal graft 212 is placed over the limb stent grafts 214a and 214b, the proximal end of the proximal graft 212 is placed in the proximal neck region 17. In this manner, the proximal graft 212 can extend aneurysm repair into the proximal neck region 17.

In some examples, the inflatable fill structure 230 can be made from PTFE, a low-durometer polyurethane, or so on. In some cases, PTFE is used for the inflatable fill structure 230 given that PTFE can be thermally bonded to the PTFE bore of the proximal graft 212 and/or the PTFE bore of the seal component 240. In some examples in which the inflatable fill structure 230 is made from PTFE, a larger inflatable fill structure 230 can implemented given that PTFE is less elastic, where such a large inflatable fill structure 230 can increase the device profile. On the other hand, in some examples in which the inflatable fill structure 230 is made from polyurethane, less material is needed as compared to the materials needed for the inflatable fill structure 230 made from PTFE given that polyurethane is more elastic than PTFE. Less material can reduce the device profile. However, polyurethane cannot be thermally bonded to the PTFE proximal graft 212 easily. Thus, if polyurethane is used for the inflatable fill structure 230, the polyurethane of the inflatable fill structure 230 is sutured to the PTFE proximal graft 212. In some cases, blood can enter a space between the bore of the proximal graft 212 and an inner lumen of the inflatable fill structure 230, thus pressurizing the inner lumen.

The inflatable fill structure 230 is fillable with a fill medium using an inflatable channel, a fill structure, or a fill line. Examples of the fill medium include but are not limited to, polyesters, PTFE, polyurethane, and so on. When the inflatable fill structure 230 is filled with the fill medium to the fullest, the inflatable fill structure 230 is in a filled or inflated state. When the inflatable fill structure 230 is not filled with any fill medium, the inflatable fill structure 230 is in an unfilled or uninflated state. The inflatable fill structure 230 surrounds at least a portion the proximal graft 212 in the inflated state. As shown, when deployed, the inflatable fill structure 230 (in the inflated state) surrounds the portion of the proximal graft 212 that is inside of the sac of the aneurysm 14 and between a lower boundary of the proximal neck region 17 and the aortic bifurcation 11. The inflatable fill structure 230 (in the inflated state) does not surround any portion of the proximal graft 212 that is inside of the proximal neck region 17. For sac management, the inflatable fill structure 230 is deployed to a location below (in the distal direction of) the seal component 240. The inflatable fill structure 230 surrounds at least the distal end of the proximal graft 212 in the inflated state. In various examples, the inflatable fill structure 230 is an endobag fixed to a portion of the external surface of proximal graft 212 and includes an outer membrane that does not extend beyond the distal end of the proximal graft 212 when the inflatable fill structure 230 is in the inflated state. In other words, the inflatable fill structure 230 (in the inflated state) does not surround any portion of the limb stent grafts 214a and 214b that is not inserted into the proximal graft 212 when the stent graft system 200 is deployed.

The inflatable fill structure 230 is fixed to the portion of the external surface of proximal graft 212 and is initially in the uninflated state when the proximal graft 212 is placed over the limb stent grafts 214a and 214b. Next, the inflatable fill structure 230 is filled with the fill medium to achieve the inflated state. A portion of the inflatable fill structure 230 extends and expands radially into a space the sac of the aneurysm 14 that is adjacent to the proximal graft 212 when the inflatable fill structure 230 is being filled. When in the uninflated state, the inflatable fill structure 230 can be confined to being around the proximal graft 212, and when in the inflated state as shown, the inflatable fill structure 230 expands radially and proximally to fill the entire (or most of the) aneurysm 14 that is between the distal end of the proximal graft 212 and the lower boundary of the proximal neck region 17. The fill medium pushes a wall (e.g., the outer membrane) of the inflatable fill structure 230 against the walls/surfaces of the aneurysm 14 when the inflatable fill structure 230 is in the filled state. When the inflatable fill structure 230 is in the filled state, the inflatable fill structure 230 can conform to the walls/surfaces of the aneurysm 14 and a portion of the outer surface of the proximal graft 212.

The proximal graft 212 and the limb stent grafts 214a and 214b are separate grafts (before deployment) that are connected, joined, or otherwise joined coupled when deployed in the manner described. Each of the proximal graft 212 and the limb stent grafts 214a and 214b is a single-lumen graft. A single-lumen graft is less complex as a structure/component and is easier and cheaper to manufacture than a bifurcated-lumen graft. In some implementations, the proximal graft 212 has a large bore diameter, which is easier to cannulate than other devices with a graft having a bifurcated lumen and thus needs less procedure time and fluoro time as compared to such other devices. The single-lumen proximal graft 212 has a bore diameter and an overall length similar to a diameter and overall length of the aorta 10, respectively. An unsupported portion of the single-lumen proximal graft 212 refers to the portion of the single-lumen proximal graft 212 that has the graft material (e.g., the PTFE) without stent for structural support. The unsupported portion of the single-lumen proximal graft 212 (outside of the docking zone 250 and above and in the proximal direction of the proximal edges/ends of the limb stent grafts 214a and 214b that are inside the single-lumen proximal graft 212 when deployed in the manner described) has a sufficient length (e.g., approximately 30 mm) for bailout procedures (such as but not limited to, deploying a Palmaz stent inside single-lumen proximal graft) or for building up from the stent graft system 200 to treat complex AAAs and TAAs.

In various arrangements, the anchor 245 (a fixation feature, a fixation stent frame, and so on) anchors, fixes, or attaches the proximal end of the stent graft system 200 (e.g., the proximal graft 212) to the walls/surfaces of the aorta 10, prevents intrusion of blood into a region between an outer wall and an inner surface of the aneurysm 14, and improves the transition from the aorta 10 into the tubular lumen of the proximal graft 212. In some examples, the anchor 245 can include a stent, graft, and/or other expandable luminal support structure. In some examples, the anchor 245 includes a suprarenal laser-cut stent with coils attached thereon. In some examples, the anchor 245 has a stent shorter than that of some current stent graft systems to eliminate free crowns. A shorter stent allows for a larger neck angle indication due to an improved stent graft flexibility. As such, the suprarenal stent of the anchor 245 is shorter and has fewer crowns and fewer anchors, allowing the stent graft systems 200 to be used for smaller treatment sizes. That is, the stent graft system 200 is a low-profile delivery system that can be used for small treatment sizes.

In some examples, the anchor 245 is a stent-like scaffold structure that can be implanted in an upper proximal opening of a tubular lumen or end of the proximal end of the proximal graft 212. As shown, the anchor 245 extends from the proximal end of the proximal graft 212 in the proximal direction. When deployed, the anchor 245 can extend from a position inside or on a boundary of the proximal neck region 17 and over the openings to the renal arteries 15 and 16 (e.g., the renal ostia). The anchor 245 includes hooks or barbs that anchor, fix, or attach to the walls/surfaces of the aorta 10 that are proximal relative to the renal ostia and the proximal neck region 17. The anchor 245 includes openings or ports to allow penetrating blood flow into the renal arteries 15 and 16. As shown, given that the anchor 245 has a stent-like scaffold structure, blood can flow into the renal arteries 15 and 16 through the renal ostia unobstructed.

Each of the grafts 212, 214a, and 214b can include one or more fill lines or inflatable channels through which hardenable inflation materials or fill polymers are communicated in liquid form. In some arrangements, each of the grafts 212, 214a, and 214b can include one or more circumferential inflatable channels extending around a circumference of a graft body of each of the grafts 212, 214a, and 214b or that may extend partially around the circumference of the graft body of each of the grafts 212, 214a, and 214b. In some implementations, the inflatable channels can be in fluid communication with each other via a longitudinal inflatable fill channel in the graft body. The network of inflatable channels can be filled with a hardenable material that hardens, cures or otherwise increases in viscosity or becomes more rigid after being injected into the channels. Hardenable inflation materials such as gels, liquids or other flowable materials that are curable to a more solid or substantially hardened state may be used to provide mechanical support to the graft body of each of the grafts 212, 214a, and 214b by virtue of the mechanical properties of the hardened material disposed within the channels. In some arrangements, the filling agent is saline. In some arrangements, the filling agent is a gas.

In some implementations, the seal component 240 (e.g., a cuff, a separate neck seal, a custom neck seal, and so on) can be an inflatable seal ring. The seal component 240 accommodates varying sizes of the aorta 10, for example, especially the varying sizes of the proximal neck region 17. In some examples and as shown in FIG. 2, the seal component 240 continuously contact an inner wall of the proximal neck region 17 to provide continuous sealing at the proximal neck region 17 while in the inflated state. Continuously contacting the inner wall of the proximal neck region 17 refers to the fact that the seal component 240, when in the inflated state, sufficiently contacts the inner wall to form a fluid seal therewith, or contacts the entire inner wall continuously, without any portion of the seal component 240 not contacting the inner wall of the proximal neck region 17.

In some implementations, the seal component 240 is coupled to the proximal graft 212. For example, the seal component 240 is attached, fixed, or otherwise coupled to the outer surface of the proximal graft 212. In the inflated state, the seal component 240 surrounds the portion of the proximal graft 212 that is in the proximal neck region 17 when the proximal graft 212 is deployed. The seal component 240 is located at or near the proximal end of the proximal graft 212. In some example, when the seal component 240 is in the inflated state, the seal component 240 does not reach and does not extend past the edge of the proximal end of the proximal graft 212 such that a portion of the proximal graft 212 adjacent to the edge of the proximal end of the proximal graft 212 is not surrounded by the seal component 240. In other examples, when the seal component 240 is in the inflated state, the seal component 240 reaches or extends past the edge of the proximal end of the proximal graft 212.

The graft materials used for the stent graft system 200 include but are not limited to, polyesters, PTFE, polyurethane, and the like. In some arrangements, each of the grafts 212, 214a, and 214b is a stent covered in the graft materials. In some arrangements the seal component 240 has or is in communication with a fill line or an inflatable channel through which hardenable inflation materials or fill polymers (e.g., polyesters, PTFE, polyurethane, and the like) are communicated in liquid form.

In some examples, the seal component 240 uses an inflatable channel and a fill port different from the inflatable channels and fill ports used by the rest of the stent graft system 200. That is, the seal component 240 does not share an inflatable channel or fill port with other components (e.g., the grafts 212, 214a, and 214b, the inflatable fill structure 230, and so on). As such, when deploying the stent graft system 200, at least a first inflatable channel coupled to the inflatable fill structure 230 and a first fill port on the inflatable fill structure 230 are used to inject fill polymers to the inflatable fill structure 230, and a second inflatable channel coupled to the seal component 240 and a second fill port of the seal component 240 are used to inject fill polymers to the seal component 240.

In some examples in which the seal component 240 is inflated using a dedicated inflatable channel that is not shared with another component (e.g., the inflatable fill structure 230) of the stent graft system 200, the seal component 240 can be inflated (using the dedicated inflatable channel) to and using a pressure higher than, for example, the pressure to which the inflatable fill structure 230 is filled using the inflatable channel of the inflatable fill structure 230. In some examples, the inflatable fill structure 230 is inflated to and using a lower pressure (e.g., approximately 120-180 mmHg), which may not be sufficient to adequately inflate the seal component 240. As the seal component 240 is filled to and using a higher pressure (e.g., 180 mm Hg-760 mm Hg), the seal component 240 can prevent the inflatable fill structure 230 from prolapsing into the renal arteries 15 and 16 when the inflatable fill structure 230 is being inflated. In that case, the seal component 240 is inflated before the inflatable fill structure 230 is inflated. The seal component 240, which is filled to a higher pressure to form the seal at the proximal neck region 17, functions like a stopper that prevents the inflatable fill structure 230 from prolapsing into the renal arteries 15 and 16 through the proximal neck region 17. Furthermore, the seal component 240 can be filled at a higher pressure because the seal component 240 is contacting healthy tissue, which is capable of handling a higher pressure for sealing and anchoring purposes. The inflatable fill structure 230 on the other hand contacts the aneurysm sac (unhealthy tissue), and therefore should be filled at a lower pressure.

In other examples, the seal component 240 can use an inflatable channel and a fill port that is also used by another component (e.g., the inflatable fill structure 230 of the stent graft system 200). That is, the seal component 240 shares an inflatable channel and a fill port with another component (e.g., the inflatable fill structure 230, and so on) of the stent graft system 200. Device profile and delivery system profile can be reduced if the inflatable channel and the fill port are shared.

In some arrangements, the seal component 240 is a wide PTFE polymer seal ring. The PTFE polymer seal ring of the seal component 240 is wider as compared to the seal rings on other devices. In one example, the seal component 240, in the inflated state and deployed entirely in the proximal neck region 17, is at least 10 mm wide along the longitudinal dimension of the aorta 10 (e.g., in the proximal distal directions). The wider seal ring improves placement accuracy given that even if the stent graft system 200 (e.g., the proximal graft 212 and the seal component 240) is placed lower (e.g., 1 mm lower) than an optimal position, the wider seal ring of the seal component 240 can nevertheless provide a sufficiently tight seal in the proximal neck region 17. The optimal position corresponds to a position of the stent graft system 200 that allows the seal component 240 (in the inflated state) to be entirely within the proximal neck region 17 (and not in the sac of the aneurysm 14) when the stent graft system 200 is deployed in the manner described. Given that the width/radius of the sac of the aneurysm 14 is larger than the width/radius of the proximal neck region 17, the portion of the seal component 240 that is outside of the proximal neck region 17 and inside of the sac of the aneurysm 14 may not form a tight seal relative to the wall of the sac. As the seal component 240 includes the wide seal ring, although the part of the seal component 240 that is outside of the proximal neck region 17 and inside of the sac of the aneurysm 14 may not form a tight seal, most of the seal component 240 is still inside of the proximal neck region 17 even if the stent graft system 200 (e.g., the proximal graft 212 and the seal component 240) is placed lower than the optimal position. The portion of the seal component 240 that is inside of the proximal neck region 17 can still provide a sufficiently tight seal. As such, even if the stent graft system 200 is placed lower than the optimal position, the placement can nevertheless be considered to be accurate because the seal component 240 can still provide the sufficiently tight seal.

Furthermore, the wider seal ring of the seal component 240 has a wider treatment diameter range. As such, a fewer number of different treatment diameter ranges of the wider seal ring are needed. This means that a fewer number of seal ring sizes and a fewer number of SKUs corresponding to those seal right sizes are needed for treating the entire vessel treatment range (e.g., to accommodate patients with different sizes of the proximal neck region 17). In one example, as soon as the seal component 240 expands radially (while being filled) to a point that the seal component 240 contacts the inner wall of the proximal neck region 17, the seal component 240 then expands longitudinally in the proximal neck region 17. This allows the seal component 240 to be applied to a larger range of blood vessel sizes. Thus, fewer sizes for the seal component 240 need to be manufactured, and flexibility and cost are improved. Furthermore, the wide seal ring can improve the neck angle indication.

As shown, the anchor 245 (for fixation or attachment to the aorta 10), the seal component 240 (for sealing the proximal neck region 17), and the inflatable fill structure 230 (for sac management) are separate components. That is, each of the anchor 245, the seal component 240, and the inflatable fill structure 230 has a single respective function, which results in a more robust design as compared to the designs of other stent graft systems.

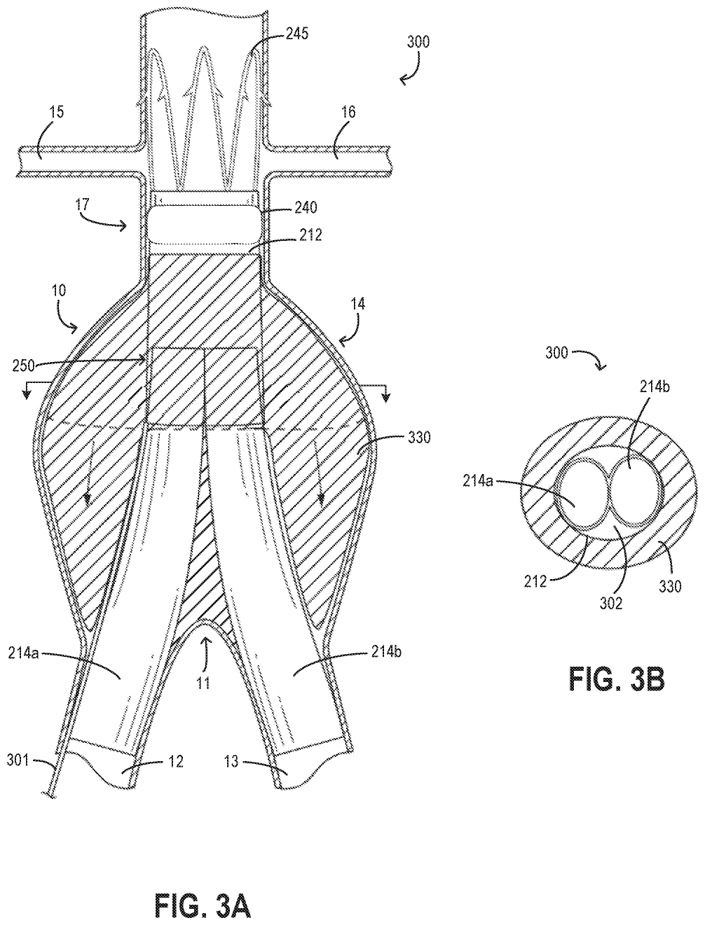

FIG. 3A is a cross-sectional view of an example stent graft system 300 deployed across the aneurysm 14 (FIG. 1) according to various arrangements. FIG. 3B is another cross-sectional view of the example stent graft system 300 (FIG. 3A) deployed across the aneurysm 14 (FIG. 1) according to various arrangements. Referring to FIGS. 1-3B, the stent graft system 300 includes the proximal graft 212, the first limb stent graft 214a, the second limb stent graft 214b, an inflatable fill structure 330, the seal component 240, and the anchor 245. The proximal graft 212, the first limb stent graft 214a, the second limb stent graft 214b, the seal component 240, and the anchor 245 are components of the stent graft system 300 that are similar to and confer similar improvements as the corresponding components of the stent graft system 200. In addition, as deployed in the aorta 10, the first limb stent graft 214a and the second limb stent graft 214b can be docked in the proximal graft 212 (e.g., in the docking zone 250) in the manner described. As shown, FIG. 3B is the cross-sectional view of the stent graft system 300 that is cut away in the docking zone 250 as shown in FIG. 3A.

In some examples, a limb diameter of the first limb stent graft 214a and a limb diameter the second limb stent graft 214b (in the docking zone 250) are substantially less than a diameter of the bore of the lumen of the proximal graft 212. In such examples, gutters 302 are typically present in the docking zone 250 as the limb stent grafts 214a and 214b are docked into the larger bore of the proximal graft 212. The inflatable fill structure 330 is shaped to seal off such gutters 302. The inflatable fill structure 330 is similar to the inflatable fill structure 230, except that the inflatable fill structure 330 is shaped to extend into the sac of the aneurysm 14 and surround each of the limb stent grafts 214a and 214b (including portions of the limb stent grafts 214a and 214b that are outside of the docking zone 250) when deployed. The inflatable fill structure 330 is filled by the fill line 301 after the limb stent grafts 214a and 214b are docked inside of the single lumen of the proximal graft 212. As shown, as being filled to the inflated state, the inflatable fill structure 330, which is fixed, bonded, attached, or otherwise coupled to the external surface of the proximal graft 212, can extend in the distal direction toward the iliac arteries 12 and 13 and the aortic bifurcation 11 to surround the limb stent grafts 214a and 214b while pushing against the surfaces/walls of the sac of the aneurysm 14 radially. The limb stent grafts 214a and 214b do not have any inflatable fill structure fixed, bonded, attached, or otherwise coupled. Accordingly, the inflatable fill structure 330 can close off the gutters 302 (by virtue of surrounding the limb stent grafts 214a and 214b) and fill the aneurysm sac from the proximal neck region 17 to aortic bifurcation 11. In some examples (not shown), the inflatable fill structure 330 can even extend into the iliac arteries 12 and 13 while surrounding the portions of the limb stent grafts 214a and 214b that are inside of the iliac arteries 12 and 13. As such, to seal the entire sac of the aneurysm 14, including the gutters 302, and sometimes even the iliac arteries 12 and 13, only one component (the inflatable fill structure 330) is needed, and only one fill line (the fill line 301) and one fill operation is needed, resulting in a shorter procedural time. Given that the limb stent grafts 214a and 214b do not have any inflatable fill structures, costs of the stent graft system 300 is also lower.

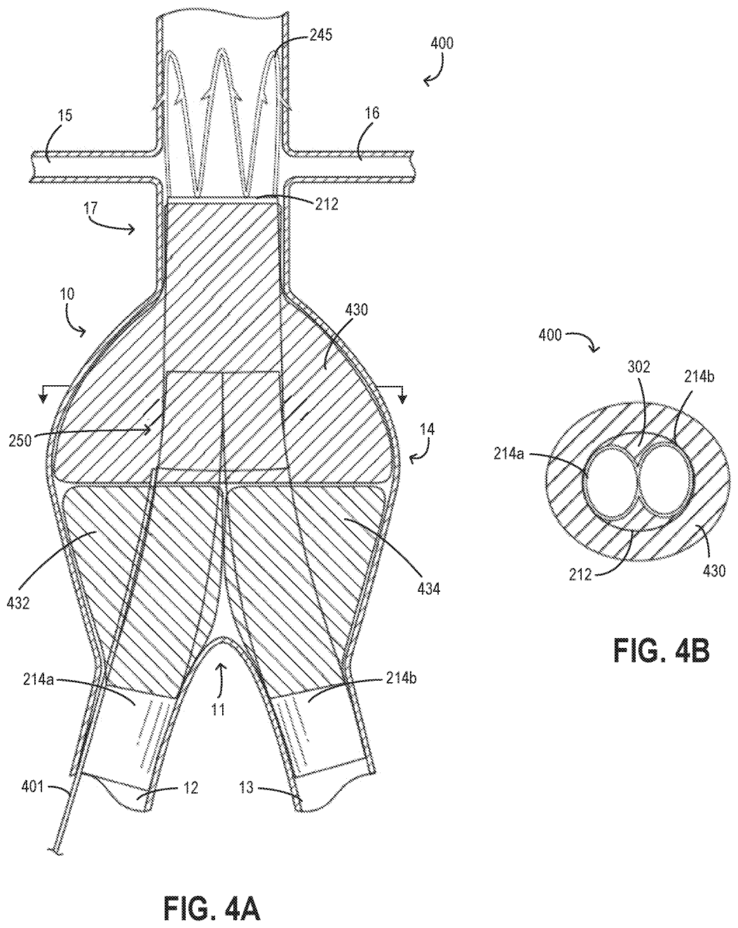

FIG. 4A is a cross-sectional view of an example stent graft system 400 deployed across the aneurysm 14 (FIG. 1) according to various arrangements. FIG. 4B is another cross-sectional view of the example stent graft system 400 (FIG. 4A) deployed across the aneurysm 14 (FIG. 1) according to various arrangements. Referring to FIGS. 1, 2, and 4A-4B, the stent graft system 400 includes the proximal graft 212, the first limb stent graft 214a, the second limb stent graft 214b, inflatable fill structures 430, 432, and 434, and the anchor 245. The proximal graft 212, the first limb stent graft 214a, the second limb stent graft 214b, and the anchor 245 are components of the stent graft system 400 that are similar to and confer similar improvements as the corresponding components of the stent graft system 200. In addition, as deployed in the aorta 10, the first limb stent graft 214a and the second limb stent graft 214b can be docked in the proximal graft 212 (e.g., in the docking zone 250) in the manner described. As shown, FIG. 4B is the cross-sectional view of the stent graft system 400 that is cut away in the docking zone 250 as shown in FIG. 4A.

The inflatable fill structure 430 is fixed, bonded, attached, or otherwise coupled to the outer surface of the proximal graft 212. In some examples, the inflatable fill structure 430 is fixed, bonded, attached, or otherwise coupled to the entire outer surface of the proximal graft 212 except for a portion of the outer surface of the proximal graft 212 that is adjacent to the edge of the proximal end of the proximal graft 212. In other examples, the inflatable fill structure 430 is fixed, bonded, attached, or otherwise coupled to the entire outer surface of the proximal graft 212. In some examples, in the inflated state, the inflatable fill structure 430 surrounds the outer surface of the proximal graft 212 (as deployed in the aorta 10), including the portion of the proximal graft 212 that is in the proximal neck region 17 and in the sac of the aneurysm 14. As such, the stent graft system 400 differs from the stent graft system 200 in that the stent graft system 400 does not include a separate seal component (e.g., the seal component 240). Instead, the inflatable fill structure 430 can provide the seal inside of the proximal neck region 17 (below or distal to the renal arteries 15 and 16). Given that the separate seal component is not provided and that a same component (e.g., the inflatable fill structure 430) provides both sealing and sac management functionalities, the stent graft system 400 is less complex and thus easier and cheaper to manufacture.

In addition, the inflatable fill structure 432 is fixed, bonded, attached, or otherwise coupled to the outer surface of the limb stent graft 214a. The inflatable fill structure 434 is fixed, bonded, attached, or otherwise coupled to the outer surface of the limb stent graft 214b. Each of the inflatable fill structures 432 and 434 can be inflated using a dedicated fill line or a fill line shared with another component of the stent graft system 400. When inflated, the inflatable fill structures 432 and 434 expand radially from the limb stent grafts 214a and 214b toward surfaces/walls of the sac of the aneurysm 14. In the inflated state, the inflatable fill structures 432 and 434 surround the limb stent grafts 214a and 214b, respectively. As shown, the inflatable fill structure 430 expands in and fills up an upper or proximal portion of the sac of the aneurysm 14 while the inflatable fill structures 432 and 434 expand in and fill up the bottom or distal portion of the sac. The entire volume of the sac is accordingly filled by the combination of the inflatable fill structures 430, 432, and 434.

In some examples, the inflatable fill structure 432 is fixed, bonded, attached, or otherwise coupled to a portion (and not an entirety) of the outer surface of the limb stent graft 214a. The inflatable fill structure 432 (when inflated) surrounds a portion (and not an entirety) of the outer surface of the limb stent graft 214a. For example, as shown, the inflatable fill structure 432 (in the inflated state) surrounds a middle portion of the limb stent graft 214a, where the middle portion is between the proximal end (the portion that is inside of the docking zone 250 when deployed) and the distal end (the portion that is inside of the iliac artery 12 when deployed) of the limb stent graft 214a. As such, the inflatable fill structure 432 is not fixed, bonded, attached, or otherwise coupled to, and does not surround, the portion of the limb stent graft 214a that is inserted into the docking zone 250 and the portion of the limb stent graft 214a that is placed in the iliac artery 12. With respect to the limb stent graft 214b, the inflatable fill structure 434 is similar to the inflatable fill structure 432.

In some examples, the inflatable fill structures 432 and 434 do not expand into the lumen of the proximal graft 212 that is in the docking zone 250 to seal off the gutters 302. If the inflatable fill structures 432 and 434 expand into the lumen of the proximal graft 212, the limb stent grafts 214a and 214b (after docking) may migrate down in the distal direction toward the aortic bifurcation 11 and out of the proximal graft 212 while the inflatable fill structures 432 and 434 are being inflated. The gutters 302 (inside of the lumen of the proximal graft 212) can be closed/sealed off by the inflated inflatable fill structure 430 after the limb stent grafts 214a and 214b are deployed inside of the proximal graft 212. In other words, the inflatable fill structure 430 (in the inflated state) fills up and seal the gutters 302 inside of the lumen of the proximal graft 212. In this manner, the proximal graft fill lumen stays connected while the proximal graft catheter is removed, in order for the ipsi limb stent grafts 214a and 214b to be deployed. In some arrangements, the portion of the proximal graft 212 that is inside of the docking zone 250 is unsupported graft (e.g., PTFE, without the stents) to conform around the limb stent grafts 214a and 214b when the limb stent grafts 214a and 214b are docked inside of the proximal graft 212.

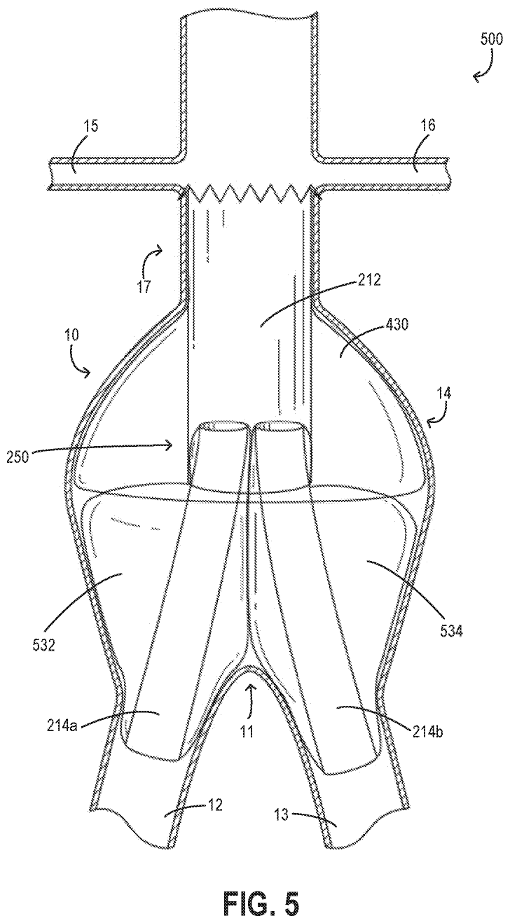

FIG. 5 is a cross-sectional view of an example stent graft system 500 deployed across the aneurysm 14 (FIG. 1) according to various arrangements. Referring to FIGS. 1, 2, and 4A-4B, and 5, the stent graft system 500 includes the proximal graft 212, the first limb stent graft 214a, the second limb stent graft 214b, the inflatable fill structure 430, inflatable fill structures 532 and 534, and the anchor 245 (not shown for clarity). The proximal graft 212, the first limb stent graft 214a, the second limb stent graft 214b, the inflatable fill structure 430, and the anchor 245 are components of the stent graft system 400 that are similar to and confer similar improvements as the corresponding components of the stent graft systems 200 and 400. In addition, as deployed in the aorta 10, the first limb stent graft 214a and the second limb stent graft 214b can be docked in the proximal graft 212 (e.g., in the docking zone 250) in the manner described.

In some examples, the inflatable fill structure 532 is fixed, bonded, attached, or otherwise coupled to the entire outer surface of the limb stent graft 214a. The inflatable fill structure 534 is fixed, bonded, attached, or otherwise coupled to the entire outer surface of the limb stent graft 214b. As such, the inflatable fill structures 532 and 534 are fixed, bonded, attached, or otherwise coupled to, and, in the inflated state, surround, in addition to the middle portion, the portions of the limb stent grafts 214a and 214b that are inserted into the docking zone 250 and the portions of the limb stent grafts 214a and 214b that are placed in the iliac artery 12.

Each of the inflatable fill structures 532 and 534 can be inflated using a dedicated fill line or a fill line shared with another component of the stent graft system 500. When inflated, the inflatable fill structures 532 and 534 expand radially from the limb stent grafts 214a and 214b toward surfaces/walls of the sac of the aneurysm 14. In the inflated state, the inflatable fill structures 532 and 534 surround the entire outer surfaces of the limb stent grafts 214a and 214b, respectively. As shown, the inflatable fill structure 430 expands in and fills up an upper or proximal portion of the sac of the aneurysm 14 while the inflatable fill structures 532 and 534 expand in and fill up the bottom or distal portion of the sac. The entire volume of the sac is accordingly filled by the combination of the inflatable fill structures 420, 532, and 534.

In some examples, the inflatable fill structures 532 and 534 expand into the lumen of the proximal graft 212 that is in the docking zone 250 to seal of the gutters in the docking zone 250. In such arrangements, the inflatable fill structure 430 can be filled in the manner described, and the delivery system for the proximal graft 212 and the inflatable fill structure 430 can be removed prior to deploying the limb stent grafts 214a and 214b and inflating the inflatable fill structures 532 and 534, thus deploy the deployment operation.

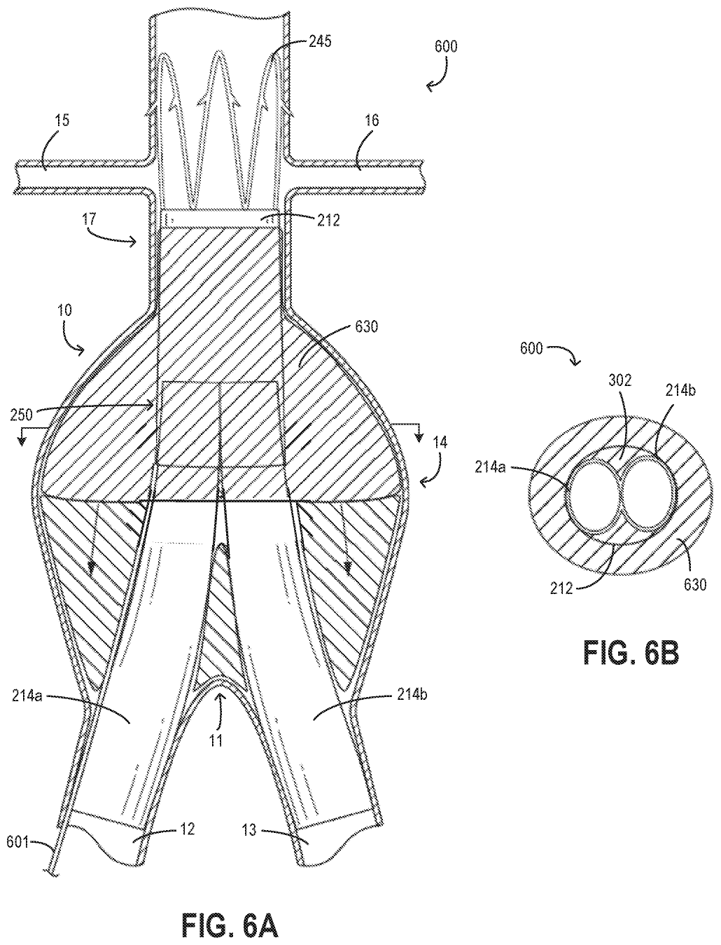

FIG. 6A is a cross-sectional view of an example stent graft system 600 deployed across the aneurysm 14 (FIG. 1) according to various arrangements. FIG. 6B is another cross-sectional view of the example stent graft system 600 (FIG. 6A) deployed across the aneurysm 14 (FIG. 1) according to various arrangements. Referring to FIGS. 1, 2, and 6A-6B, the stent graft system 600 includes the proximal graft 212, the first limb stent graft 214a, the second limb stent graft 214b, an inflatable fill structure 630, and the anchor 245. The proximal graft 212, the first limb stent graft 214a, the second limb stent graft 214b, and the anchor 245 are components of the stent graft system 600 that are similar to and confer similar improvements as the corresponding components of the stent graft system 200. In addition, as deployed in the aorta 10, the first limb stent graft 214a and the second limb stent graft 214b can be docked in the proximal graft 212 (e.g., in the docking zone 250) in the manner described. As shown, FIG. 6B is the cross-sectional view of the stent graft system 600 that is cut away in the docking zone 250 as shown in FIG. 6A.

The inflatable fill structure 630 is fixed, bonded, attached, or otherwise coupled to the outer surface of the proximal graft 212. In some examples, the inflatable fill structure 630 is fixed, bonded, attached, or otherwise coupled to the entire outer surface of the proximal graft 212 except for a portion of the outer surface of the proximal graft 212 that is adjacent to the edge of the proximal end of the proximal graft 212. In other examples, the inflatable fill structure 630 is fixed, bonded, attached, or otherwise coupled to the entire outer surface of the proximal graft 212. In some examples, in the inflated state, the inflatable fill structure 630 surrounds the outer surface of the proximal graft 212 (as deployed in the aorta 10), including the portion of the proximal graft 212 that is in the proximal neck region 17 and in the sac of the aneurysm 14. As such, the stent graft system 600 differs from the stent graft system 200 in that the stent graft system 600 does not include a separate seal component (e.g., the seal component 240). Instead, the inflatable fill structure 630 can provide the seal inside of the proximal neck region 17 (below or distal to the renal arteries 15 and 16).

In addition, the inflatable fill structure 630 is shaped to seal off the gutters 302. The inflatable fill structure 630 is shaped to extend into the sac of the aneurysm 14 and surround each of the limb stent grafts 214a and 214b (including portions of the limb stent grafts 214a and 214b that are outside of the docking zone 250) when deployed. The inflatable fill structure 630 is filled by the fill line 601 after the limb stent grafts 214a and 214b are docked inside of the single lumen of the proximal graft 212. As shown, as being filled to the inflated state, the inflatable fill structure 630, which is fixed, bonded, attached, or otherwise coupled to the external surface of the proximal graft 212, can extend in the distal direction toward the iliac arteries 12 and 13 and the aortic bifurcation 11 to surround the limb stent grafts 214a and 214b while pushing against the surfaces/walls of the sac of the aneurysm 14 radially. The limb stent grafts 214a and 214b do not have any inflatable fill structure fixed, bonded, attached, or otherwise coupled. Accordingly, the inflatable fill structure 630 can close off the gutters 302 (by virtue of surrounding the limb stent grafts 214a and 214b) and fill the aneurysm sac from the proximal neck region 17 to aortic bifurcation 11. In some examples (not shown), the inflatable fill structure 630 can even extend into the iliac arteries 12 and 13 while surrounding the portions of the limb stent grafts 214a and 214b that are inside of the iliac arteries 12 and 13.

As such, to seal the entire sac of the aneurysm 14, including the gutters 302, the proximal neck region 17, and sometimes even the iliac arteries 12 and 13, only one component (the inflatable fill structure 630) is needed, and only one fill line (the fill line 601) and one fill operation is needed to perform both sealing and sac management functionalities, resulting in a shorter procedural time. Given that the limb stent grafts 214a and 214b do not have any inflatable fill structures, complexity and costs of the stent graft system 600 are also lower.

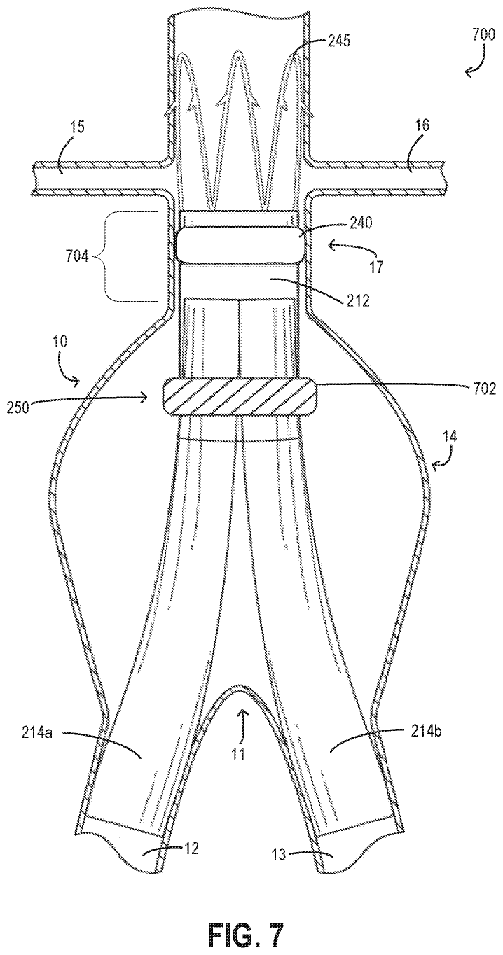

FIG. 7 is a cross-sectional view of an example stent graft system 700 deployed across the aneurysm 14 (FIG. 1) according to various arrangements. Referring to FIGS. 1, 2, and 7, the stent graft system 700 includes the proximal graft 212, the first limb stent graft 214a, the second limb stent graft 214b, the seal component 240, the anchor 245, and a support component 702. The proximal graft 212, the first limb stent graft 214a, the second limb stent graft 214b, the seal component 240, and the anchor 245 are components of the stent graft system 700 that are similar to and confer similar improvements as the corresponding components of the stent graft system 200. In addition, as deployed in the aorta 10, the first limb stent graft 214a and the second limb stent graft 214b can be docked in the proximal graft 212 (e.g., in the docking zone 250) in the manner described.

An unsupported section 704 of the proximal graft 212 has the graft material (e.g., the PTFE) without stent for structural support. The unsupported section 704 is configured for proximal extension. That is, the unsupported section 704 extends into the proximal neck region 17 when the proximal graft 212 is deployed within the aorta 10 in the manner described. The seal component 240 is attached, fixed, or otherwise coupled to the outer surface of the unsupported section 704 of the proximal graft 212.

In some arrangements, the support component 702 is a support ring or balloon made from a polymer (e.g., PTFE, polyurethane, and so on). The support component 702 surrounds the portion of the proximal graft 212 that is in the docking zone 250. In other words, the support component 702 is attached, fixed, bonded (e.g., thermally bonded), sutured, or otherwise coupled to the proximal graft 212, e.g., on the outer surface of the proximal graft 212. In some examples, the portion of the proximal graft 212 that is in the docking zone 250 is unsupported. In some examples, the entire proximal graft 212 (including the docking zone 250 and the unsupported section 704) are unsupported. The support component 702 can facilitate in cannulating the proximal graft 212 prior to or as an inflatable fill structure (not shown) of the proximal graft 212 is being filled via a suitable fill line. Such an inflatable fill structure can be fixed, bonded, attached, or otherwise coupled to the outer surface of the proximal graft 212. In some examples, in the inflated state, such an inflatable fill structure surrounds the outer surface of the proximal graft 212 (as deployed in the aorta 10), including one or more of the portion of the proximal graft 212 that is in the proximal neck region 17, the portion of the proximal graft 212 in the sac of the aneurysm 14, the gutters, and so on. The support component 702 can be a support inflatable fill structure that is inflated to provide structural integrity to the unsupported proximal graft 212 (e.g., the portion that is in the docking zone 250), before or while the inflatable fill structure is inflated. The support component 702, in the inflated state, provides structural integrity by preventing collapse of the proximal graft 212. In some examples, the support component 702 can be integrated with the delivery system that delivers the proximal graft 212. That is, the support component 702 can be filled using the catheter (a shared fill line) used to fill the proximal graft 212. Alternatively, the support component 702 can be filled using a catheter separate from the catheter used for the delivery system for the proximal graft 212. The support component 702 does not increase the device profile.

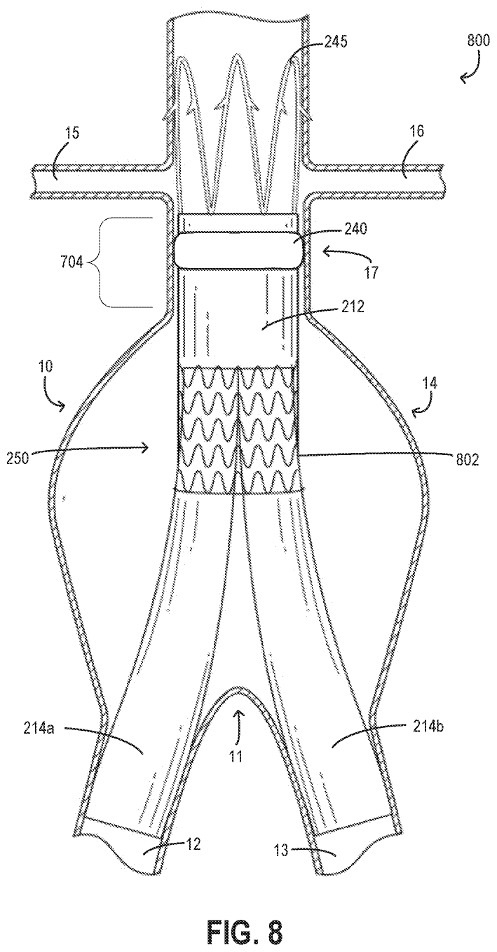

FIG. 8 is a cross-sectional view of an example stent graft system 800 deployed across the aneurysm 14 (FIG. 1) according to various arrangements. Referring to FIGS. 1, 2, 7, and 8, the stent graft system 800 includes the proximal graft 212, the first limb stent graft 214a, the second limb stent graft 214b, the seal component 240, and the anchor 245. The proximal graft 212, the first limb stent graft 214a, the second limb stent graft 214b, the seal component 240, and the anchor 245 are components of the stent graft system 800 that are similar to and confer similar improvements as the corresponding components of the stent graft system 200. In addition, as deployed in the aorta 10, the first limb stent graft 214a and the second limb stent graft 214b can be docked in the proximal graft 212 (e.g., in the docking zone 250) in the manner described. As described, the unsupported section 704 of the proximal graft 212 has the graft material (e.g., the PTFE) without stent for structural support. In some examples, in addition to the unsupported section 704, other portions of the proximal graft 212 may be unsupported. In some examples, the entirety of the proximal graft 212 is unsupported. The seal component 240 is attached, fixed, or otherwise coupled to the outer surface of the unsupported section 704 of the proximal graft 212.

In some arrangements, the proximal graft 212 includes a wire-wound stent component 802 embedded therein. In some examples, the wire-wound stent component 802 includes wire-wound stents (having multiple wire-wound rings) and does not have any graft material coupled thereto, such that the lumen of the proximal graft 212 is open at the wire-wound stent component 802 for easy cannulation. In other examples, the wire-wound stent component 802 has graft material coupled thereto. The wire-wound stent component 802 is located at the distal end of the proximal graft 212 in some examples. The wire-wound stent component 802 is located in the docking zone 250 of the proximal graft 212 in some examples.

The wire-wound stent component 802 can facilitate cannulating the proximal graft 212 prior to or as an inflatable fill structure (not shown) of the proximal graft 212 is being filled via a suitable fill line. Such an inflatable fill structure can be fixed, bonded, attached, or otherwise coupled to the outer surface of the proximal graft 212. In some examples, in the inflated state, such an inflatable fill structure surrounds the outer surface of the proximal graft 212 (as deployed in the aorta 10), including one or more of the portion of the proximal graft 212 that is in the proximal neck region 17, the portion of the proximal graft 212 that is in the sac of the aneurysm 14, the gutters, and so on. The wire-wound stent component 802 can provide structural integrity to the proximal graft 212, before or while the inflatable fill structure is inflated. The wire-wound stent component 802 provides structural integrity by preventing collapse of the proximal graft 212 and avoiding kinking the lumen of the proximal graft 212 in angulated anatomy. The wire-wound stent component 802 can provide improved mechanical locking between the proximal graft 212 and the limb stent grafts 214a and 214b in the docking zone 250 by providing a sufficient radial force to minimize the likelihood of dislodgement between the proximal graft 212 and the limb stent grafts 214a and 214b, thus improving joint separation resistance and minimizing Type III Endoleaks.

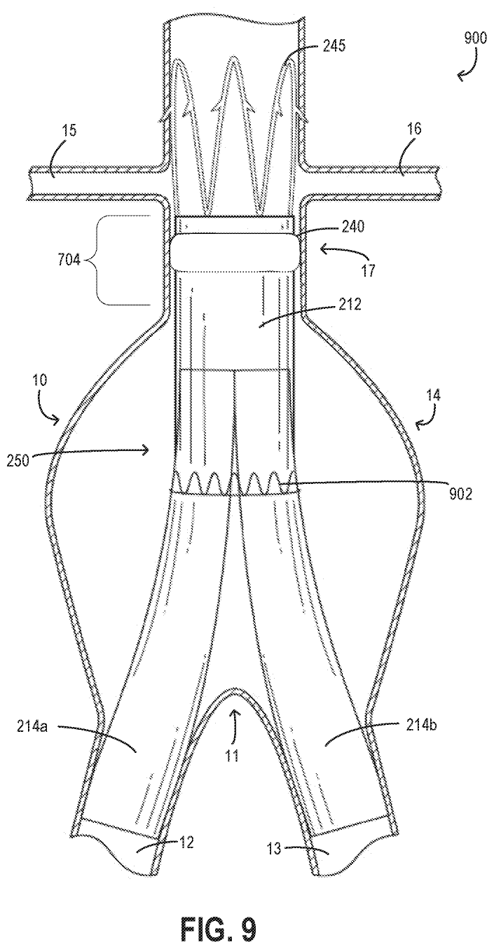

FIG. 9 is a cross-sectional view of an example stent graft system 900 deployed across the aneurysm 14 (FIG. 1) according to various arrangements. Referring to FIGS. 1, 2, and 7-9, the stent graft system 900 includes the proximal graft 212, the first limb stent graft 214a, the second limb stent graft 214b, the seal component 240, and the anchor 245. The proximal graft 212, the first limb stent graft 214a, the second limb stent graft 214b, the seal component 240, and the anchor 245 are components of the stent graft system 900 that are similar to and confer similar improvements as the corresponding components of the stent graft system 200. In addition, as deployed in the aorta 10, the first limb stent graft 214a and the second limb stent graft 214b can be docked in the proximal graft 212 (e.g., in the docking zone 250) in the manner described. As described, the unsupported section 704 of the proximal graft 212 has the graft material (e.g., the PTFE) without stent for structural support. In some examples, in addition to the unsupported section 704, other portions of the proximal graft 212 may be unsupported. In some examples, the entirety of the proximal graft 212 is unsupported. The seal component 240 is attached, fixed, or otherwise coupled to the outer surface of the unsupported section 704 of the proximal graft 212.

In some arrangements, the proximal graft 212 includes a wire-wound stent ring 902 embedded therein. In some examples, the wire-wound stent ring 902 includes a single ring of wire-wound stent and does not have any graft material coupled thereto, such that the lumen of the proximal graft 212 is open at the wire-wound stent component 902 for easy cannulation. In other examples, the wire-wound stent ring 902 has graft material coupled thereto. The wire-wound stent ring 902 is located at the distal end of the proximal graft 212 and abuts an edge of the proximal graft 212 in some examples. The wire-wound stent ring 902 is located in the docking zone 250 of the proximal graft 212 in some examples.

The wire-wound stent ring 902 can facilitate cannulating the proximal graft 212 prior to or as an inflatable fill structure (not shown) of the proximal graft 212 is being filled via a suitable fill line. Such an inflatable fill structure can be fixed, bonded, attached, or otherwise coupled to the outer surface of the proximal graft 212. In some examples, in the inflated state, such an inflatable fill structure surrounds the outer surface of the proximal graft 212 (as deployed in the aorta 10), including one or more of the portion of the proximal graft 212 that is in the proximal neck region 17, the portion of the proximal graft 212 that is in the sac of the aneurysm 14, the gutters, and so on. The wire-wound stent ring 902 can provide structural integrity to the proximal graft 212, before or while the inflatable fill structure is inflated. The wire-wound stent ring 902 provides structural integrity by preventing collapse of the proximal graft 212 and avoiding kinking the lumen of the proximal graft 212 in angulated anatomy. The wire-wound stent ring 902 can provide improved mechanical locking between the proximal graft 212 and the limb stent grafts 214a and 214b in the docking zone 250 by providing a sufficient radial force to minimize the likelihood of dislodgement between the proximal graft 212 and the limb stent grafts 214a and 214b, thus improving joint separation resistance and minimizing Type III Endoleaks.

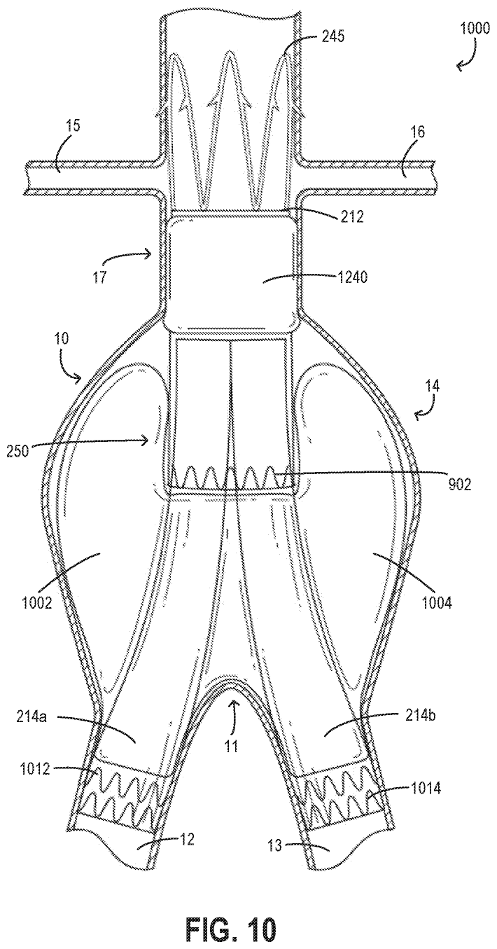

FIG. 10 is a cross-sectional view of an example stent graft system 1000 deployed across the aneurysm 14 (FIG. 1) according to various arrangements. Referring to FIGS. 1, 2, and 10, the stent graft system 1000 includes the proximal graft 212, the first limb stent graft 214a, the second limb stent graft 214b, a seal component 1240, the anchor 245, an inflatable fill structure 1002, and an inflatable fill structure 1004. The proximal graft 212, the first limb stent graft 214a, the second limb stent graft 214b, and the anchor 245 are components of the stent graft system 1000 that are similar to and confer similar improvements as the corresponding components of the stent graft system 200. In some arrangements, the seal component 1240 is similar to the seal component 240, except that the seal component 1240 is wider such that a portion of the seal component 1240 extends outside of the proximal neck region 17 and inside of the sac of the aneurysm 14. As deployed in the aorta 10, the first limb stent graft 214a and the second limb stent graft 214b can be docked in the proximal graft 212 (e.g., in the docking zone 250) in the manner described. In some examples, the portion of the proximal graft 212 that is in the docking zone 250 includes the wire-wound stent ring 902. In some examples, the wire-wound stent ring 902 includes a single ring of wire-wound stent and does not have any graft material coupled thereto, such that the lumen of the proximal graft 212 is open at the wire-wound stent component 902 for easy cannulation.

In some arrangements, each of the limb stent grafts 214a and 214b includes a respective one of wire-wound stent components 1012 and 1014 embedded therein. In some examples, each of the wire-wound stent components 1012 and 1014 includes wire-wound stents (having multiple wire-wound rings) and does not have any graft material coupled thereto, such that the lumen of each of the limb stent grafts 214a and 214b is open at the respective one of the wire-wound stent components 1012 and 1014 for easy cannulation. In other examples, the wire-wound stent components 1012 and 1014 have graft material coupled thereto. Each of the wire-wound stent components 1012 and 1014 is located at the distal end of the respective one of the limb stent grafts 214a and 214b in some examples and is placed in the iliac arteries 12 and 13 when deployed.

The inflatable fill structure 1002 is fixed, bonded, attached, or otherwise coupled to at least a portion of the outer surface of the limb stent graft 214a. The inflatable fill structure 1004 is fixed, bonded, attached, or otherwise coupled to at least a portion of the outer surface of the limb stent graft 214b. Each of the inflatable fill structures 1002 and 1004 can be inflated using a dedicated fill line or a fill line shared with another component of the stent graft system 1000. When inflated, the inflatable fill structures 1002 and 1004 expand radially from the limb stent grafts 214a and 214b toward surfaces/walls of the sac of the aneurysm 14. In the inflated state, the inflatable fill structures 1002 and 1004 surround the limb stent grafts 214a and 214b, respectively.

In some examples, the inflatable fill structure 1002 is fixed, bonded, attached, or otherwise coupled to a portion (and not an entirety) of the outer surface of the limb stent graft 214a. The inflatable fill structure 1002 (when inflated) surrounds a portion (and not an entirety) of the outer surface of the limb stent graft 214a. In some arrangements, the inflatable fill structure 1002 is not fixed, bonded, attached, or otherwise coupled to, and does not surround, the portion of the limb stent graft 214a that is placed in the iliac artery 12 when deployed or the portion of the limb stent graft 214a corresponding to the wire-wound stent components 1012 and 1014. In other arrangements, the inflatable fill structure 1002 is fixed, bonded, attached, or otherwise coupled to, and surrounds, the portion of the limb stent graft 214a that is placed in the iliac artery 12 when deployed or the portion of the limb stent graft 214a corresponding to the wire-wound stent components 1012 and 1014. With respect to the limb stent graft 214b, the inflatable fill structure 1004 is similar to the inflatable fill structure 1002. In some arrangements, the inflatable fill structures 1002 and 1004 are not fixed, bonded, attached, or otherwise coupled to, and do not surround, the portions of the respective ones of the limb stent grafts 214a and 214b that are inserted into the docking zone 250. In some examples, the inflatable fill structures 1002 and 1004 surround the stent grafts 214a and 214b that are outside of the docking zone 250 contacting the edge of the distal end of the proximal graft 212 to seal off the gutters. In some examples, the inflatable fill structures 1002 and 1004 expand into the lumen of the proximal graft 212 that is in the docking zone 250 to seal off the gutters.

Furthermore, the inflatable fill structures 1002 and 1004 are shaped to extend into the sac of the aneurysm 14 and surround the portion of the proximal graft 212 that is in the sac when deployed. As shown, while being filled to the inflated state, the inflatable fill structures 1002 and 1004 can extend in the proximal direction toward the proximal neck region 17 to surround the proximal graft 212 while pushing against the surfaces/walls of the sac of the aneurysm 14 radially. The proximal graft 212 does not have any inflatable fill structure fixed, bonded, attached, or otherwise coupled. Given that no inflatable fill structure is provided for the stent graft, the stent graft system 1000 is cheaper to manufacture. As shown, the entire volume of the sac is accordingly filled by the inflatable fill structures 1002 and 1004.

In some examples, instead of the two inflatable fill structures 1002 and 1004, a single inflatable fill structure fixed, bonded, attached, or otherwise coupled to either one or both of the limb stent grafts 214a and 214b can be used to surround the limb stent grafts 214a and 214b as well as to extend into the sac of the aneurysm 14 and surround the portion of the proximal graft 212 that is in the sac when deployed.

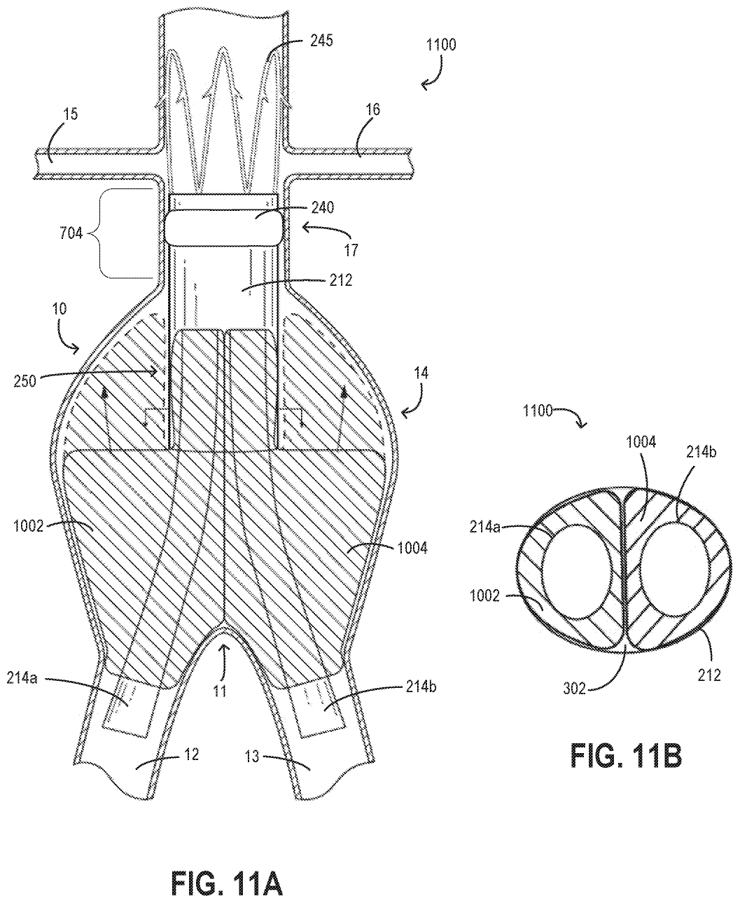

FIG. 11A is a cross-sectional view of an example stent graft system 1100 deployed across the aneurysm 14 (FIG. 1) according to various arrangements. FIG. 11B is another cross-sectional view of the example stent graft system 1100 (FIG. 11A) deployed across the aneurysm 14 (FIG. 1) according to various arrangements. Referring to FIGS. 1, 2, and 10-11B, the stent graft system 1100 includes the proximal graft 212, the first limb stent graft 214a, the second limb stent graft 214b, the seal component 240, the anchor 245, and the inflatable fill structures 1002 and 1004. The proximal graft 212, the first limb stent graft 214a, the second limb stent graft 214b, the seal component 240, and the anchor 245 are components of the stent graft system 1100 that are similar to and confer similar improvements as the corresponding components of the stent graft system 200. In addition, the inflatable fill structures 1002 and 1004 are components of the stent graft system 1100 that are similar to and confer similar improvements as the corresponding components of the stent graft system 1000. The stent graft system 1100 is different from the stent graft system 1000 in that the proximal graft 212 of the stent graft system 1100 does not include the wire-wound stent component 902, and the limb stent grafts 214a and 214b of the stent graft system 1100 do not include the wire-wound stent components 1012 and 1014. As described, the unsupported section 704 of the proximal graft 212 has the graft material (e.g., the PTFE) without stent for structural support.

As described, the inflatable fill structures 1002 and 1004 are fixed, bonded, attached, or otherwise coupled to and surround at least portions of the outer surface of the limb stent grafts 214a and 214b. In the examples in which the inflatable fill structures 1002 and 1004 are not fixed, bonded, attached, or otherwise coupled to, and do not surround, the portions of the respective ones of the limb stent grafts 214a and 214b that are inserted into the docking zone 250, the inflatable fill structures 1002 and 1004 are shaped to extend into the sac of the aneurysm 14 and surround the portion of the proximal graft 212 that is in the sac when deployed. In other arrangements, the inflatable fill structures 1002 and 1004 are fixed, bonded, attached, or otherwise coupled to, and surround, the portions of the respective ones of the limb stent grafts 214a and 214b that are inserted into the docking zone 250. In such arrangements, when being inflated by a dedicated or shared fill line, each of the inflatable fill structures 1002 and 1004 can expand within the lumen of the proximal graft 212 to seal off the gutters 302.

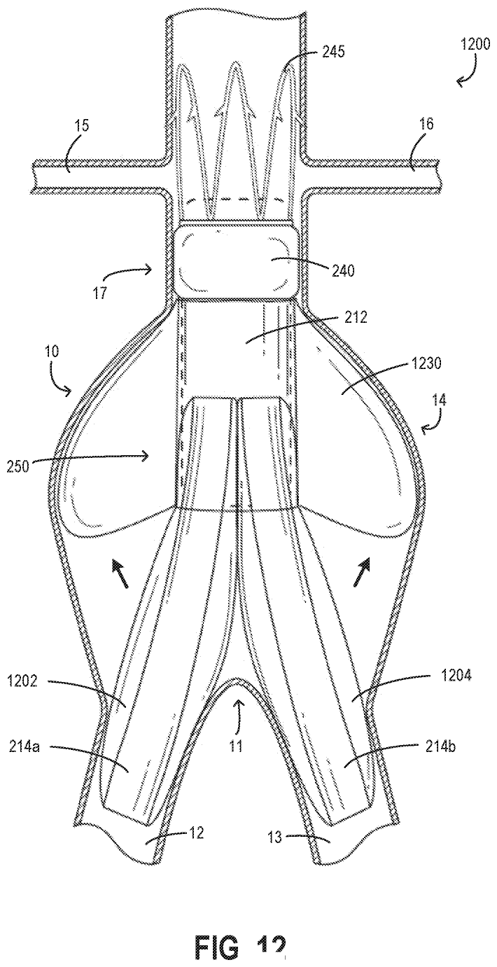

FIG. 12 is a cross-sectional view of an example stent graft system 1200 deployed across the aneurysm 14 (FIG. 1) according to various arrangements. Referring to FIGS. 1, 2, and 12, the stent graft system 1200 includes the proximal graft 212, the first limb stent graft 214a, the second limb stent graft 214b, the seal component 240, the anchor 245, and inflatable fill structures 1202, 1204, and 1230. The proximal graft 212, the first limb stent graft 214a, the second limb stent graft 214b, the seal component 240, and the anchor 245 are components of the stent graft system 1200 that are similar to and confer similar improvements as the corresponding components of the stent graft system 200.

In some examples, a shape and compliance (elasticity) of the inflatable fill structure 1230 allow the inflatable fill structure 1230 to form a funnel when inflated within the sac of the aneurysm 14. For example, the inflatable fill structure 1230 is similar to the inflatable fill structure 230, except that the inflatable fill structure 1230, when being filled, expands radially toward the surfaces/walls of the sac of the aneurysm 14 and also in the distal direction toward the iliac arteries 12 and 13, such that the portion of the inflatable fill structure 1230 abutting and adjacent to the surfaces/walls of the sac of the aneurysm 14 extend (e.g., along the surfaces/walls of the sac of the aneurysm 14) farther in the distal direction as compared to the potions of the of the inflatable fill structure 1230 abutting and adjacent to the proximal graft 212, thus creating the funnel shape. The inflatable fill structure 1230 is made from material that is sufficiently soft and elastic to allow the inflatable fill structure 1230 to form the funnel shape.

The funnel shape is used to facilitate cannulation. In one example, the proximal graft 212 can be deployed within the aorta 10 in the manner described. The inflatable fill structure 1230 can be inflated to form the funnel shape. The limb stent grafts 214a and 214b can be inserted into the lumen of the proximal graft 212 as guided by the funnel shape of the inflatable fill structure 1230. That is, the sloped surface of the inflatable fill structure 1230 can guide the proximal ends of the limb stent grafts 214a and 214b into the lumen of the proximal graft 212 as the limb stent grafts 214a and 214b move in the proximal direction toward the proximal neck region 17. In another example, the limb stent grafts 214a and 214b can be deployed within the aorta 10 in the manner described. While the proximal graft 212 is being inserted into the aorta 10, the inflatable fill structure 1230 can be inflated to form the funnel shape. The sloped surface of the of the inflatable fill structure 1230 can guide the proximal graft 212 so that the limb stent grafts 214a and 214b can be inserted into the lumen of the proximal graft 212 as the proximal graft 212 move in the distal direction. In some examples, the stent graft delivery system uses an integrated contra wire instead of cannulating retrograde into the large bore of the proximal graft 212.

In some examples, the seal component 240 can be made from a material (e.g., polyesters, PTFE, polyurethane, and so on) that is less compliant than the material (e.g., PTFE, a low-durometer polyurethane, and so on) from which the inflatable fill structure 1230 is made. The less compliant seal component 240 (approximately 1 cm in width) can provide a tighter seal in the proximal neck region 17.

The inflatable fill structure 1202 is fixed, bonded, attached, or otherwise coupled to the entire outer surface of the limb stent graft 214a (including the portions of the limb stent graft 214a that is placed in the iliac artery 12 and in the docking zone 250) when deployed. The inflatable fill structure 1204 is fixed, bonded, attached, or otherwise coupled to the entire outer surface of the limb stent graft 214b (including the portions of the limb stent graft 214b that is placed in the iliac artery 13 and in the docking zone 250) when deployed. Each of the inflatable fill structures 1202 and 1204 can be inflated using a dedicated fill line or a fill line shared with another component of the stent graft system 1200. When inflated, the inflatable fill structures 1202 and 1204 expand radially from the limb stent grafts 214a and 214b toward surfaces/walls of the sac of the aneurysm 14. As such, the entire volume of the sac is accordingly filled by the combination of the inflatable fill structures 1202, 1204, and 1230. In the inflated state, the inflatable fill structures 1202 and 1204 surround the limb stent grafts 214a and 214b, respectively. The inflatable fill structures 1202 and 1204 can also expand within the lumen of the proximal graft 212 to seal off any gutters therein. Furthermore, the inflatable fill structures 1202 and 1204 can expand within the iliac arties 12 and 13 to form a seal in the iliac arties 12 and 13 when the limb stent grafts 214a and 214b are deployed.

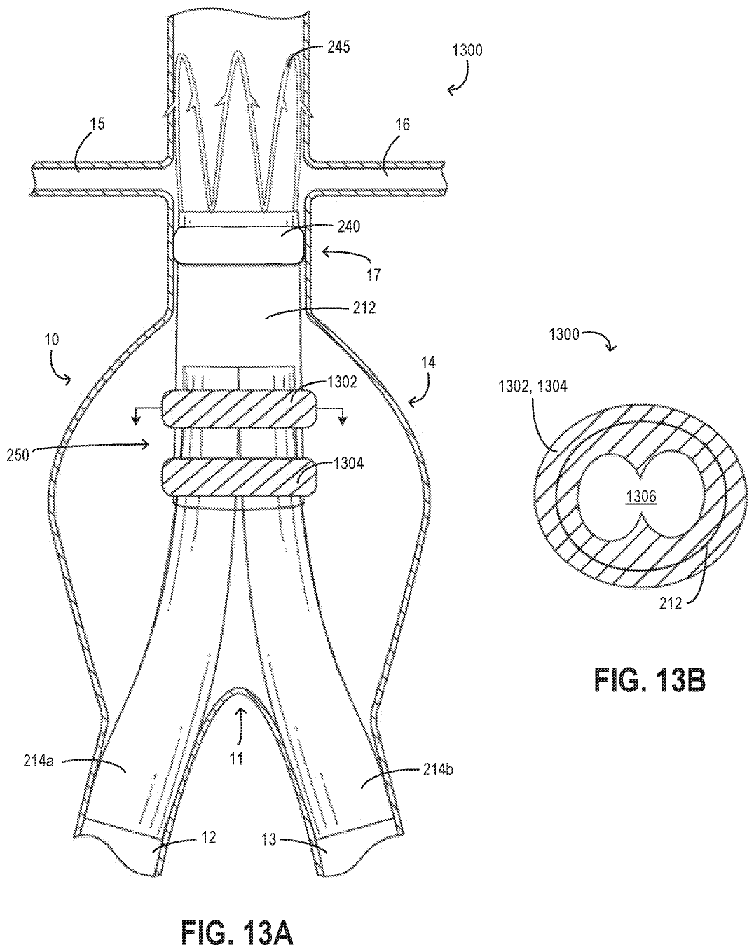

FIG. 13A is a cross-sectional view of an example stent graft system 1300 deployed across the aneurysm 14 (FIG. 1) according to various arrangements. FIG. 13B is another cross-sectional view of the example stent graft system 1300 (FIG. 13A) deployed across the aneurysm 14 (FIG. 1) according to various arrangements. Referring to FIGS. 1, 2, 13A, and 13B, the stent graft system 1300 includes the proximal graft 212, the first limb stent graft 214a, the second limb stent graft 214b, the seal component 240, the anchor 245, and at least one support components (e.g., support components 1302 and 1304). The proximal graft 212, the first limb stent graft 214a, the second limb stent graft 214b, the seal component 240, and the anchor 245 are components of the stent graft system 1300 that are similar to and confer similar improvements as the corresponding components of the stent graft system 200. In addition, as deployed in the aorta 10, the first limb stent graft 214a and the second limb stent graft 214b can be docked in the proximal graft 212 (e.g., in the docking zone 250) in the manner described.

To seal any gutters that may form in the lumen of the proximal graft 212 when the limb stent grafts 214a and 214b are inserted in the lumen of the proximal graft 212, the at least one support components (e.g., the support components 1302 and 1304) are embedded in the proximal graft 212. In some arrangements, the support components 1302 and 1304 are support inflatable fill structures such as but not limited to, support rings or balloons made from a polymer (e.g., PTFE, polyurethane, and so on). The support components 1302 and 1304 are embedded in the portion of the proximal graft 212 that is in the docking zone 250. The support components 1302 and 1304 are attached, fixed, bonded (e.g., thermally bonded), sutured, or otherwise coupled to the proximal graft 212 such that an interior portion (including an inner surface portion) of each of the support components 1302 and 1304 is inside of the lumen of the proximal graft 212 while the remaining exterior portion (including an outer surface portion) of the support components 1302 and 1304 is outside of the proximal graft 212. In some examples, the portion of the proximal graft 212 that is in the docking zone 250 is unsupported.

After the proximal graft 212 is deployed in the aorta 10 in the manner described, each of the support components 1302 and 1304 can be inflated using a dedicated fill line or a shared fill line shared with another component of the stent graft system 1300. In some examples, each of the support components 1302 and 1304 can be pre-shaped using a bi-lobe balloon on the catheter used to deploy the proximal graft 212, where the support components 1302 and 1304 are inflated around the bi-lobe balloon on the catheter. Accordingly, in the inflated state, each of the support components 1302 and 1304 forms an opening 1306 (commensurate with the shape of the bi-lobe balloon on the catheter) through which the limb stent grafts 214a and 214b can be inserted. The opening 1306 appears to be a bi-lobe opening. Given that the support components 1302 and 1304 are elastic, and the opening 1306 is slightly smaller than the sum of the cross-section area of the proximal ends of the limb stent grafts 214a and 214b, the support components 1302 and 1304 form tight seals around the limb stent grafts 214a and 214b when inserted. While two support components 1302 and 1304 are shown, one or three or more support components such as but not limited to, the support components 1302 and 1304 can be implemented.

The implementation of the support components 1302 and 1304 allows the stent graft system 1300 to seal off the gutters without needing inflatable fill structures such as endobags. If there are no type II Endoleaks present, physicians may select the stent graft system 1300 given that not filling the entire sac of the aneurysm 14 with polymer (e.g., the endobags) is preferred.

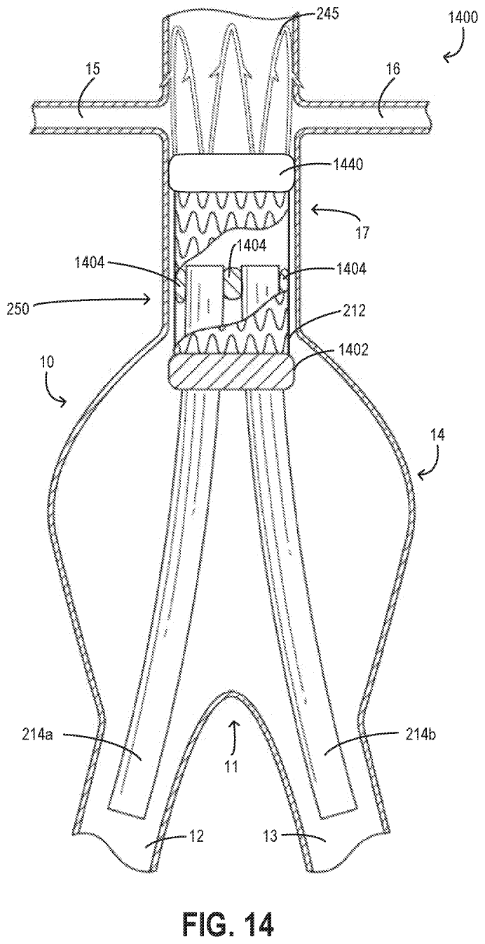

FIG. 14 is a cross-sectional view of an example stent graft system 1400 deployed across the aneurysm 14 (FIG. 1) according to various arrangements. Referring to FIGS. 1, 2, and 14, the stent graft system 1400 includes the proximal graft 212, the first limb stent graft 214a, the second limb stent graft 214b, seal components 1402 and 1440, the anchor 245, and at least one internal support components (e.g., the internal support component 1404). The proximal graft 212, the first limb stent graft 214a, the second limb stent graft 214b, and the anchor 245 are components of the stent graft system 1400 that are similar to and confer similar improvements as the corresponding components of the stent graft system 200. In some examples, the anchor 245 of the stent graft system 1400 can be fixed or attached to the proximal end of the proximal graft 212 or to the seal component 1440. The seal component 1440 is similar to the seal component 240, except that the seal component 1440 is narrower as compared to the seal component 240 in some arrangements. In addition, as deployed in the aorta 10, the first limb stent graft 214a and the second limb stent graft 214b can be docked in the proximal graft 212 (e.g., in the docking zone 250) in the manner described. As shown, a portion of the docking zone 250 is in the proximal neck region 17 while the remaining portion of the docking zone 250 is in the sac of the aneurysm 14. The proximal graft 212 is shown to have wire-wound stents (having multiple wire-wound rings) in addition to the graft material (e.g., the proximal graft 212 in FIG. 14 is a stent graft).

In some examples, the seal component 1402 is coupled to the distal end of the proximal graft 212 to seal the gutters formed when the limb stent grafts 214a and 214b are inserted into the lumen of the proximal graft 212 in the docking zone 250. The seal component 1402 can be an inflatable fill structure made from a polymer (e.g., PTFE, polyurethane, and so on) that can be inflated using a dedicated fill line or a shared fill line shared with another component of the stent graft system 1400. In the inflated state, the seal component 1402 may have a single bi-lobe opening or two openings to receive the proximal ends of the limb stent grafts 214a and 214b. Given the elasticity of the material of the seal component 1402, the seal component 1402 forms a seal around the limb stent grafts 214a and 214b at the lumen opening of the proximal graft 212.

To provide additional sealing features to seal the gutters that may form in the lumen of the proximal graft 212 when the limb stent grafts 214a and 214b are inserted in the lumen of the proximal graft 212, the internal support component 1404 is embedded in the proximal graft 212. In some arrangements, the internal support component 1404 is a support inflatable fill structure such as but not limited to, an endobag made from a polymer (e.g., PTFE, polyurethane, and so on). The internal support component 1404 is embedded in the portion of the proximal graft 212 that is in the docking zone 250. The internal support component 1404 is attached, fixed, bonded (e.g., thermally bonded), sutured, or otherwise coupled to the internal surface of the proximal graft 212. The internal surface of the proximal graft 212 faces the lumen of the proximal graft 212. The internal support component 1404 expands within the lumen of the proximal graft 212 when filled.

After the proximal graft 212 is deployed in the aorta 10 in the manner described, the limb stent grafts 214a and 214b are inserted into the lumen of the proximal graft 212. The internal support component 1404 can be inflated using a dedicated fill line or a shared fill line shared with another component of the stent graft system 1400 after the limb stent grafts 214a and 214b are inserted. In the inflated state, the internal support component 1404 forms a seal around the proximal ends of the limb stent grafts 214a and 214b, including a space between the limb stent grafts 214a and 214b and a space between the inner surface of the proximal graft 212 and each of the limb stent grafts 214a and 214b, as shown. Given that the internal support component 1404 is elastic (e.g., more compliant than a polymer support ring such as the support components 1302 and 1304), and that the internal support component 1404 is inflated inward within the lumen of the proximal graft 212, the internal support component 1404 can form a tight seal around the limb stent grafts 214a and 214b when inserted. While one internal support component 1404 is shown, two or more internal support components such as but not limited to, the internal support component 1404 can be implemented.

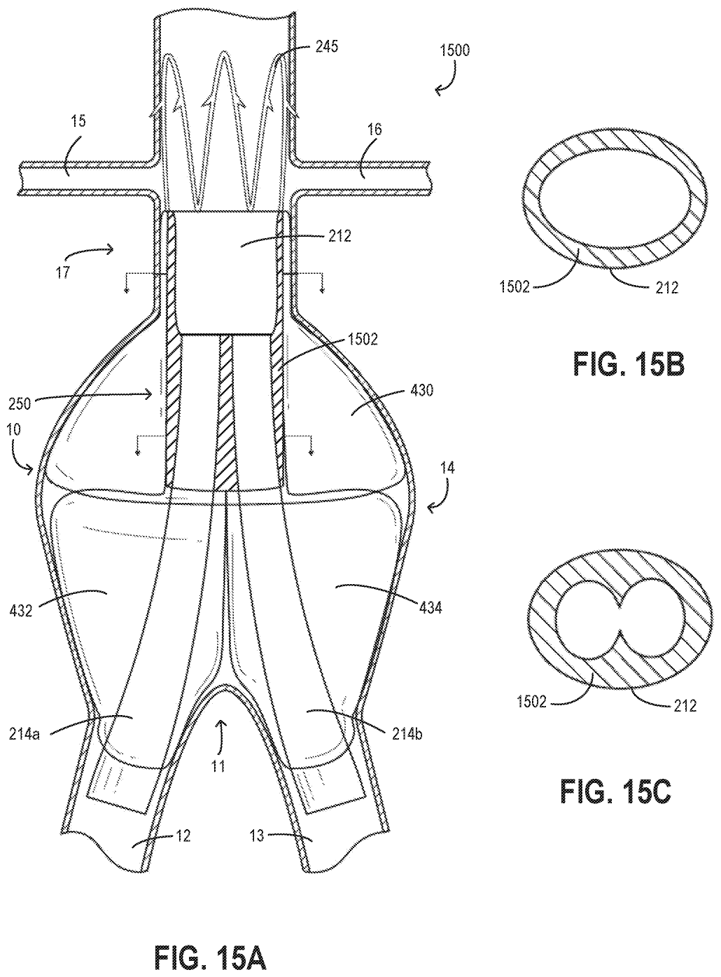

FIG. 15A is a cross-sectional view of an example stent graft system 1500 deployed across the aneurysm 14 (FIG. 1) according to various arrangements. FIG. 15B is another cross-sectional view of the example stent graft system 1500 (FIG. 15A) deployed across the aneurysm 14 (FIG. 1) according to various arrangements. FIG. 15C is yet another cross-sectional view of the example stent graft system 1500 (FIG. 15A) deployed across the aneurysm 14 (FIG. 1) according to various arrangements. Referring to FIGS. 1, 2, 4A-4B, and 15A-15C the stent graft system 1500 includes the proximal graft 212, the first limb stent graft 214a, the second limb stent graft 214b, the inflatable fill structures 430, 432, and 434, the anchor 245, and an internal inflatable fill structure 1502. The proximal graft 212, the first limb stent graft 214a, the second limb stent graft 214b, and the anchor 245 are components of the stent graft system 1500 that are similar to and confer similar improvements as the corresponding components of the stent graft system 200. As deployed in the aorta 10, the first limb stent graft 214a and the second limb stent graft 214b can be docked in the proximal graft 212 (e.g., in the docking zone 250) in the manner described.

In addition, the inflatable fill structures 430, 432, and 434 are components of the stent graft system 1500 that are similar to and confer similar improvements as the corresponding components of the stent graft system 400, except that the inflatable fill structure 430 (in the inflated state) does not fills up or seal the gutters inside of the lumen of the proximal graft 212. Instead, the internal inflatable fill structure 1502 can be inflated to seal the gutters.

For example, to seal the gutters that may form in the lumen of the proximal graft 212 when the limb stent grafts 214a and 214b are inserted in the lumen of the proximal graft 212, the internal inflatable fill structure 1502 is embedded in the proximal graft 212. In some arrangements, the internal inflatable fill structure 1502 is a support inflatable fill structure such as but not limited to, an endobag made from a polymer (e.g., PTFE, polyurethane, and so on). The internal inflatable fill structure 1502 is attached, fixed, bonded (e.g., thermally bonded), sutured, or otherwise coupled to the entire internal surface of the proximal graft 212. The internal surface of the proximal graft 212 faces the lumen of the proximal graft 212. The internal inflatable fill structure 1502 expands within the lumen of the proximal graft 212 when filled.

In some examples, in the inflated state, the internal inflatable fill structure 1502 includes a proximal portion (the cross section of which is shown in FIG. 15B) corresponding to the proximal end of the proximal graft 212 and a distal portion (the cross section of which is shown in FIG. 15C) corresponding to the distal end of the proximal graft 212. The distal portion of the internal inflatable fill structure 1502 corresponds to the docking zone 250. When filled, the proximal portion of the internal inflatable fill structure 1502 forms a large lumen while the distal portion of the internal inflatable fill structure 1502 forms a bi-lobe lumen. The internal inflatable fill structure 1502 can be pre-shaped by a catheter used to deploy the proximal graft 212. For example, after the proximal graft 212 is deployed in the aorta 10 in the manner described, the proximal portion of the internal inflatable fill structure 1502 is inflated around a balloon of the catheter having a circular or oval cross section while the distal portion of the internal inflatable fill structure 1502 is inflated around a bi-lobe balloon of the catheter. As such, the internal inflatable fill structure 1502 form a bifurcated lumen within the lumen of the proximal graft 212. The internal inflatable fill structure 1502 can be inflated using a dedicated fill line or a shared fill line shared with another component of the stent graft system 1500 before the limb stent grafts 214a and 214b are inserted.

In the inflated state, the distal portion of the internal inflatable fill structure 1502 forms a seal around the proximal ends of the limb stent grafts 214a and 214b, including a space between the limb stent grafts 214a and 214b and a space between the inner surface of the proximal graft 212 and each of the limb stent grafts 214a and 214b, as shown. Given that the internal inflatable fill structure 1502 is elastic, and that the internal support component 1404 is inflated inward within the lumen of the proximal graft 212, the internal inflatable fill structure 1502 can form a tight seal around the limb stent grafts 214a and 214b when inserted.

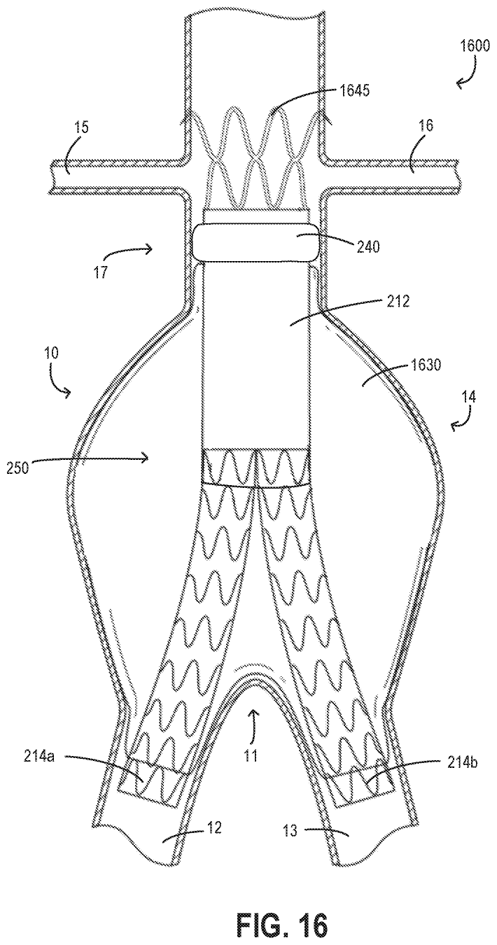

FIG. 16 is a cross-sectional view of an example stent graft system 1600 deployed across the aneurysm 14 (FIG. 1) according to various arrangements. Referring to FIGS. 1, 2, and 16, the stent graft system 1600 includes the proximal graft 212, the first limb stent graft 214a, the second limb stent graft 214b, the seal component 240, an inflatable fill structure 1630, and an anchor 1645. The proximal graft 212, the first limb stent graft 214a, the second limb stent graft 214b, and the seal component 240 are components of the stent graft system 1600 that are similar to and confer similar improvements as the corresponding components of the stent graft system 200. The anchor 1645 is similar to the anchor 245, except that the anchor 1645 includes wire-wound stents having multiple wire-wound rings. The anchor 1645 includes hooks or barbs on the wire-wound stents that anchor, fix, or attach to the walls/surfaces of the aorta 10 that are proximal relative to the renal ostia and the proximal neck region 17. As deployed in the aorta 10, the first limb stent graft 214a and the second limb stent graft 214b can be docked in the proximal graft 212 (e.g., in the docking zone 250) in the manner described. The limb stent grafts 214a and 214b are shown to include wire-wound stents having multiple wire-wound rings in some examples.

The inflatable fill structure 1630 is fixed, bonded, attached, or otherwise coupled to the outer surface of the proximal graft 212. In some examples, the inflatable fill structure 1630 is fixed, bonded, attached, or otherwise coupled to the entire outer surface of the proximal graft 212 except for a portion of the outer surface of the proximal graft 212 that is adjacent to the edge of the proximal end of the proximal graft 212. In other examples, the inflatable fill structure 1630 is fixed, bonded, attached, or otherwise coupled to the entire outer surface of the proximal graft 212.

The inflatable fill structure 1630 is a bifurcated inflatable fill structure or endobag, such that in the inflated state, the inflatable fill structure 1630 surrounds the outer surface of the proximal graft 212 (as deployed in the aorta 10) while providing two lumens for receiving limb stent grafts 214a and 214b. The inflatable fill structure 1630 can be pre-shaped by a catheter used to deploy the proximal graft 212. For example, after the proximal graft 212 is deployed in the aorta 10 in the manner described, the inflatable fill structure 1630 is inflated around a bifurcated balloon of the catheter to shape the lumens for receiving limb stent grafts 214a and 214b, while the inflatable fill structure 1630 expands radially toward the surfaces/walls of the sac of the aneurysm 14 to fill up the entire sac except for the lumen of the proximal graft 212 and the bifurcated balloon. The inflatable fill structure 1630 can be inflated using a dedicated fill line or a shared fill line shared with another component of the stent graft system 1600 before the limb stent grafts 214a and 214b are inserted. After, the limb stent grafts 214a and 214b can be inserted into the lumens of the inflatable fill structure 1630 and the lumen of the proximal graft 212. The lumens of the inflatable fill structure 1630 lead to and are in communication with the lumen of the proximal graft 212. The inflatable fill structure 1630 can surround the limb stent grafts 214a and 214b and provide a tight seal, including in the area around the docking zone 250 to seal the gutters. Given that the inflatable fill structure 1630 can seal the gutters while filling up the entire sac, thus only one polymer fill step is needed in the stent graft system 1600. The limb stent grafts 214a and 214b also do not need additional inflatable fill structures coupled thereto, thus reducing complexity and cost.

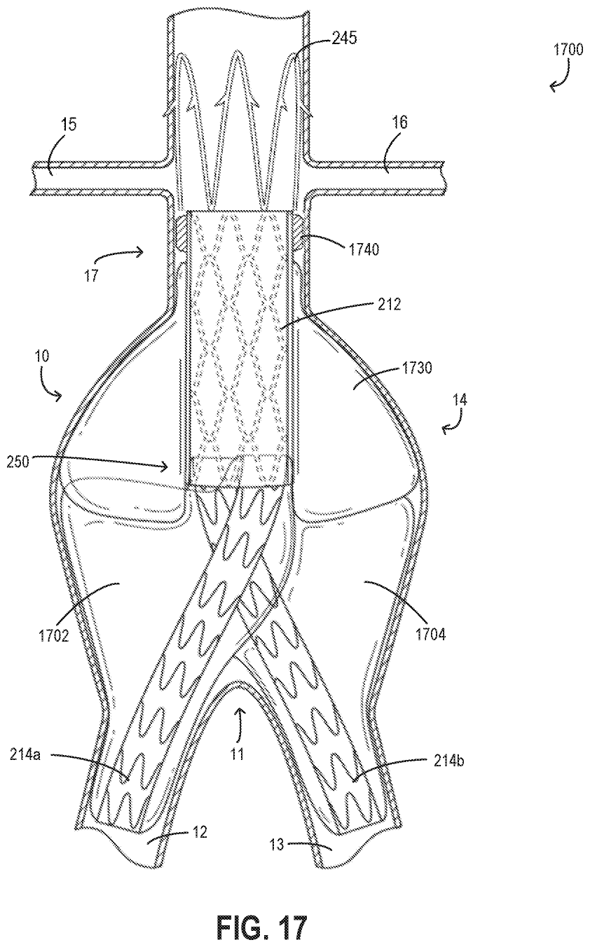

FIG. 17 is a cross-sectional view of an example stent graft system 1700 deployed across the aneurysm 14 (FIG. 1) according to various arrangements. Referring to FIGS. 1, 2, and 17, the stent graft system 1700 includes the proximal graft 212, the first limb stent graft 214a, the second limb stent graft 214b, a seal component 1740, the anchor 245, and inflatable fill structures 1702, 1704, and 1730. The proximal graft 212, the first limb stent graft 214a, the second limb stent graft 214b, and the anchor are components of the stent graft system 1700 that are similar to and confer similar improvements as the corresponding components of the stent graft system 200. As shown, each of the stent grafts 214a and 214b includes stents having multiple rings. In some examples, the stent grafts 214a and 214b include Nellix stents. As deployed in the aorta 10, the first limb stent graft 214a and the second limb stent graft 214b can be docked in the proximal graft 212 (e.g., in the docking zone 250) in the manner described.

As shown, the proximal graft 212 includes a laminated stent component such as but not limited to, Teflon-laminated nickel-titanium (NiTi)-stents. The laminated stent component prevents the lumen of the proximal graft 212 from kinking and collapsing in angulated anatomies and during polymer filling of the inflatable fill structure 1730, which may be soft. Providing the laminated stent component eliminates the need for a support balloon on the delivery system that delivers the proximal graft 212 into the aorta 10, thus resulting in reduced cost and reduced profile.

The seal component 1740 is similar to the seal component 240, except that the seal component 1740 is narrower as compared to the seal component 240 in some arrangements. In some examples, the seal component 1740 can be made from a material (e.g., polyesters, PTFE, polyurethane, and so on) that is less compliant than the material (e.g., PTFE, a low-durometer polyurethane, and so on) from which the inflatable fill structure 1730 is made.

The less compliant and more rigid seal component 1740 (approximately 1 cm in width) can provide a tighter seal in the proximal neck region 17 and a more defined edge than a soft endobag. The more defined edge at the proximal end of the proximal graft 212 can improve proximal placement accuracy.

The inflatable fill structure 1730 is fixed, bonded, attached, or otherwise coupled to at least a portion of the outer surface of the proximal graft 212. In some examples, the inflatable fill structure 1730 is fixed, bonded, attached, or otherwise coupled to the entire outer surface of the proximal graft 212 except for a portion of the outer surface of the proximal graft 212 that is coupled to the seal component 1740. In some examples, in the inflated state, the inflatable fill structure 1730 surrounds the outer surface of the proximal graft 212 (as deployed in the aorta 10), including a portion of the proximal graft 212 that is in the proximal neck region 17 and in the sac of the aneurysm 14.

The inflatable fill structure 1702 is fixed, bonded, attached, or otherwise coupled to the entire outer surface of the limb stent graft 214a (including the portions of the limb stent graft 214a that is placed in the iliac artery 12 and in the docking zone 250) when deployed. The inflatable fill structure 1704 is fixed, bonded, attached, or otherwise coupled to the entire outer surface of the limb stent graft 214b (including the portions of the limb stent graft 214b that is placed in the iliac artery 13 and in the docking zone 250) when deployed. Each of the inflatable fill structures 1702 and 1704 can be inflated using a dedicated fill line or a fill line shared with another component of the stent graft system 1700. When inflated, the inflatable fill structures 1702 and 1704 expand radially from the limb stent grafts 214a and 214b toward surfaces/walls of the sac of the aneurysm 14. As such, the entire volume of the sac is accordingly filled by the combination of the inflatable fill structures 1702, 1704, and 1730. In The inflatable fill structures 1702 and 1704 can also expand within the lumen of the proximal graft 212 to seal off any gutters therein. Furthermore, the inflatable fill structures 1702 and 1704 can expand within the iliac arties 12 and 13 to form a seal in the iliac arties 12 and 13 when the limb stent grafts 214a and 214b are deployed.

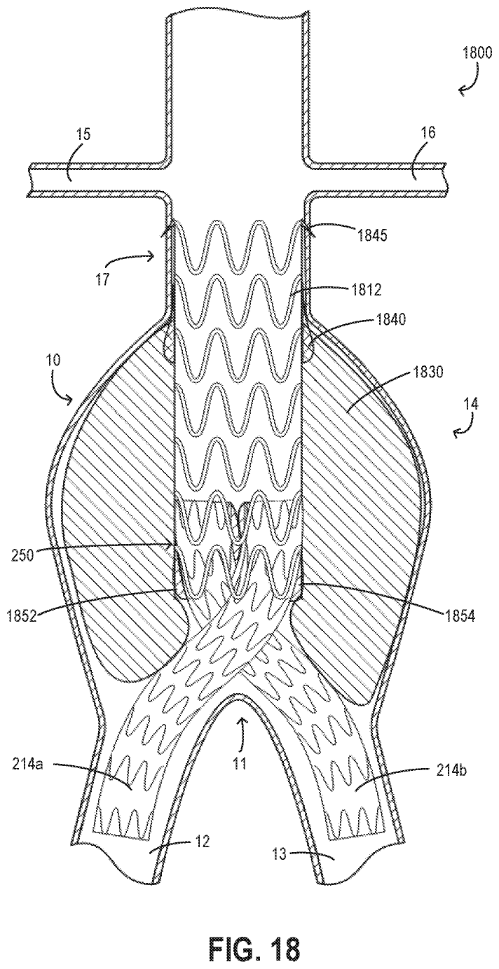

FIG. 18 is a cross-sectional view of an example stent graft system 1800 deployed across the aneurysm 14 (FIG. 1) according to various arrangements. Referring to FIGS. 1, 2, and 18, the stent graft system 1800 includes a laminated stent component 1812, the first limb stent graft 214a, the second limb stent graft 214b, a seal component 1840, an anchor 1845, locking features 1852 and 1854, and inflatable fill structure 1830. The first limb stent graft 214a and the second limb stent graft 214b are components of the stent graft system 1800 that are similar to and confer similar improvements as the corresponding components of the stent graft system 200. As shown, each of the stent grafts 214a and 214b includes stents having multiple rings. In some examples, the stent grafts 214a and 214b include Nellix stents. As deployed in the aorta 10, the first limb stent graft 214a and the second limb stent graft 214b can be docked in the laminated stent component 1812 (e.g., in the docking zone 250) in the manner described.

As shown, the laminated stent component 1812 includes a component such as but not limited to, Teflon-laminated Nickel-Titanium (NiTi)-stents. The laminated stent can be wire-wound or laser-cut. The laminated stent component prevents the lumen of the laminated stent component 1812 from kinking and collapsing in angulated anatomies and during polymer filling of the inflatable fill structure 1830, which may be soft. Providing the laminated stent component eliminates the need for a support balloon on the delivery system that delivers the laminated stent component 1812 into the aorta 10, thus resulting in reduced cost and reduced profile. After the inflatable fill structure 1830 is filled, the limbs stent grafts 214a and 214b (which may be Nellix) are inserted into the laminated stent component 1812 in the docking zone 250.

The inflatable fill structure 1830 is fixed, bonded, attached, or otherwise coupled to at least a portion of the outer surface of the laminated stent component 1812. In some examples, the inflatable fill structure 1830 is fixed, bonded, attached, or otherwise coupled to the entire outer surface of the proximal graft 212 except for a portion of the outer surface of the laminated stent component 1812 that is coupled to the seal component 1840. In some examples, in the inflated state, the inflatable fill structure 1830 surrounds the outer surface of the laminated stent component 1812 (as deployed in the aorta 10), including a portion of the laminated stent component 1812 that is in the proximal neck region 17 and in the sac of the aneurysm 14. In some arrangements, in the inflated state and when deployed, the inflatable fill structure 1830 surrounds or encapsulate the outer surface of a portion of the seal component 1840 that is inside of the sac of the aneurysm 14. In some examples, the inflatable fill structure 1830 extends in the distal direction toward the aortic bifurcation 11 and the iliac arteries 12 and 13 to fill up the entirety of the sac of the aneurysm 14.

In some example, the anchor 1845 can be hooks or barbs on the stents of the laminated stent component 1812. As shown, the hooks or barbs of the anchor 1845 are located on the stent ring that is closest to the renal arteries 15 and 16. The hooks or barbs of the anchor 1845 can be located on another stent ring of the laminated stent component 1812 as well as on more than one stent ring of the laminated stent component 1812.

The seal component 1840 can be an inflatable seal ring. In some implementations, the seal component 1840 is coupled to the laminated stent component 1812. For example, the seal component 1840 is attached, fixed, or otherwise coupled to the outer surface of the proximal end of the laminated stent component 1812. In the inflated state, the seal component 240 surrounds the portion of the laminated stent component 1812 that is in the proximal neck region 17 and in the sac of the aneurysm 14 when the laminated stent component 1812 is deployed. In some example, when the seal component 1840 is in the inflated state, the seal component 1840 does not reach and does not extend past the edge of the proximal end of the laminated stent component 1812 such that a portion (e.g., the portion with the anchor 1845) of the laminated stent component 1812 adjacent to the edge of the proximal end of the laminated stent component 1812 is not surrounded by the seal component 1840. Generally, a device having a seal component of a certain width can be deployed in a range of neck lengths of the aortic neck region 17, meaning that a seal component having an inflated width longer than the neck length cannot be deployed in the aortic neck region 17 of a subject having that neck length. On the other hand, the stent graft systems described herein (e.g., the stent graft system 1800) can be deployed in the aorta 10 of subjects having a neck length that is shorter than the neck lengths deployable by other devices. This is because if the neck length of the aortic neck region 17 is short, the seal component 1840 is configured to extend into the sac of the aneurysm 14 (while the anchor 1845 is fixed to the wall of the aortic neck region 17) when the seal component 1840 does not otherwise have any space to expand in the aortic neck region 17. The portion of the seal component 1840 that is in the sac of the aneurysm 14 can be used in conjunction with the inflatable fill structure 1830 (e.g., the inflatable fill structure 1830 encapsulate the portion the seal component 1840 that is in the sac) for sac management.

Each of the locking features 1852 and 1854 includes a polymer seal sack on the proximal ends of a respective one of the limbs stent grafts 214a and 214b. In some arrangements, the polymer seal sack is an inflatable fill structure that is fixed, bonded, attached, or otherwise coupled to the outer surface of a respective one of the limb stent grafts 214a and 214b that is in the docking zone 250 when deployed. The locking features 1852 and 1854 (when inflated) surrounds the outer surface of the proximal end of a respective one of the limb stent grafts 214a and 214b. In some arrangements, the locking features 1852 and 1854 are not fixed, bonded, attached, or otherwise coupled to, and does not surround, the portion of the respective one of the limb stent grafts 214a and 214b that is outside of the docking zone 250 when deployed. When inflated by a dedicated fill line or a shared fill line while the limb stent grafts 214a and 214b are docked in the docking zone 250, the locking features 1852 and 1854 expand radially from the proximal ends of the limb stent grafts 214a and 214b toward the lumen of the laminated stent component 1812 to seal the gutters between the inner surface of the laminated stent component 1812 and the outer surface of the limb stent grafts 214a and 214b that are in the docking zone 250.

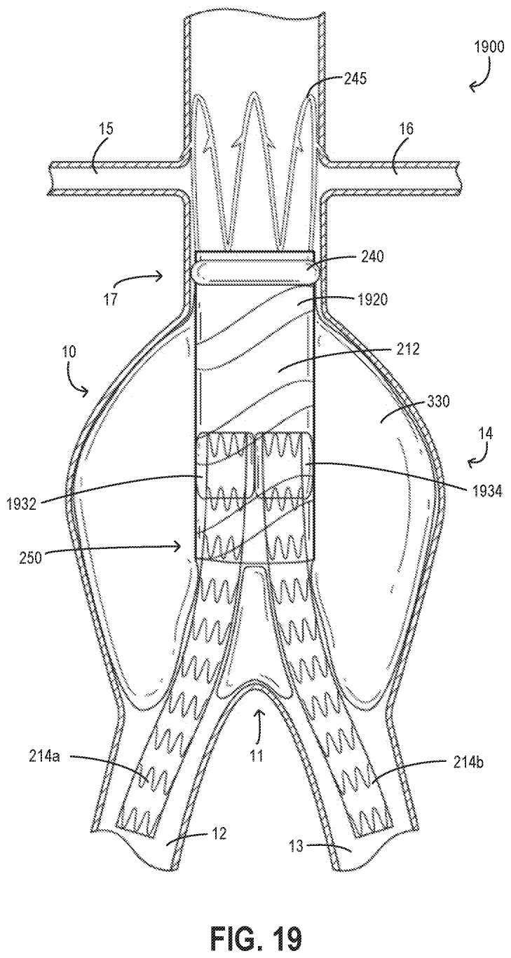

FIG. 19 is a cross-sectional view of an example stent graft system 1900 deployed across the aneurysm 14 (FIG. 1) according to various arrangements. Referring to FIGS. 1-3B and 19, the stent graft system 1900 includes the proximal graft 212, the first limb stent graft 214a, the second limb stent graft 214b, the inflatable fill structure 330, the seal component 240, the anchor 245, a support structure 1920, inflatable structures 1932 and 1934. The proximal graft 212, the first limb stent graft 214a, the second limb stent graft 214b, the seal component 240, and the anchor 245 are components of the stent graft system 1900 that are similar to and confer similar improvements as the corresponding components of the stent graft system 200. The inflatable fill structure 330 is a component of the stent graft system 1900 that are similar to and confer similar improvements as the corresponding components of the stent graft system 300. As deployed in the aorta 10, the first limb stent graft 214a and the second limb stent graft 214b can be docked in the proximal graft 212 (e.g., in the docking zone 250) in the manner described.

The stent graft system 1900 differs from the stent graft system 300 in that the proximal graft 212 includes the support structure 1920. The support structure 1920 is embedded in the proximal graft 212. In some arrangements, the support structure 1920 include helix-shaped polymer support rings. The support structure 1920 is attached, fixed, bonded (e.g., thermally bonded), sutured, or otherwise coupled to the internal surface of the proximal graft 212. The support structure 1920 faces or in the lumen of the proximal graft 212. The support structure 1920 expands within the lumen of the proximal graft 212 when filled by a dedicated fill line or a shared fill line shared with another component of the of the stent graft system 1900. The support structure 1920, prevents the lumen of the proximal graft 212 from kinking and collapsing in angulated anatomies and during polymer filling of the inflatable fill structure 330, which may be soft. The helix shape of the support structure 1920 can also improve the joint integrity of the docked limb stent grafts 214a and 214b.

In some examples, the inflatable fill structure 1932 is fixed, bonded, attached, or otherwise coupled to the outer surface of the proximal end of the limb stent graft 214a. The inflatable fill structure 1932 (when inflated) surrounds the outer surface of the proximal end of the limb stent graft 214a. In some arrangements, the inflatable fill structure 1002 is not fixed, bonded, attached, or otherwise coupled to, and does not surround, the portion of the limb stent graft 214a that is outside of the docking zone 250 when deployed. With respect to the limb stent graft 214b, the inflatable fill structure 1934 is similar to the inflatable fill structure 1932. When filled using a dedicated fill line or a shared fill line shared with another component of the of the stent graft system 1900 while the proximal ends of the limb stent grafts 214a and 214b are docked into the lumen of the proximal graft 212 in the docking zone 250, the inflatable fill structures 1932 and 1934 expand from within the lumen of the proximal graft 212 that is in the docking zone 250 to seal off the gutters. The inflatable fill structures 1932 and 1934 expand radially from the proximal ends of the limb stent grafts 214a and 214b toward the inner surface of the proximal graft 212. As described, the inflatable fill structure 330, in the inflated state, can fill up the sac of the aneurysm 14.

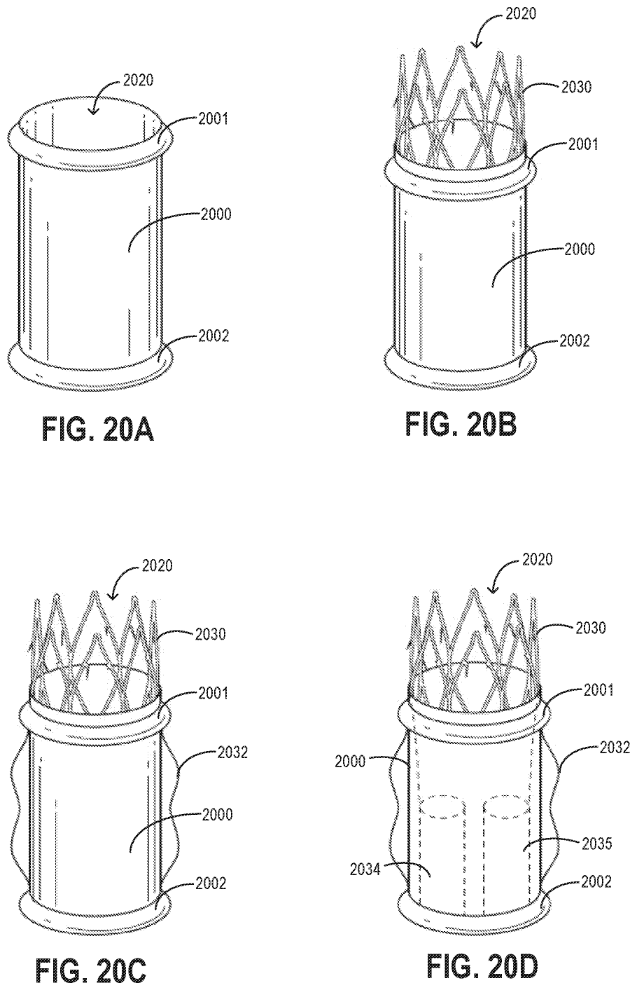

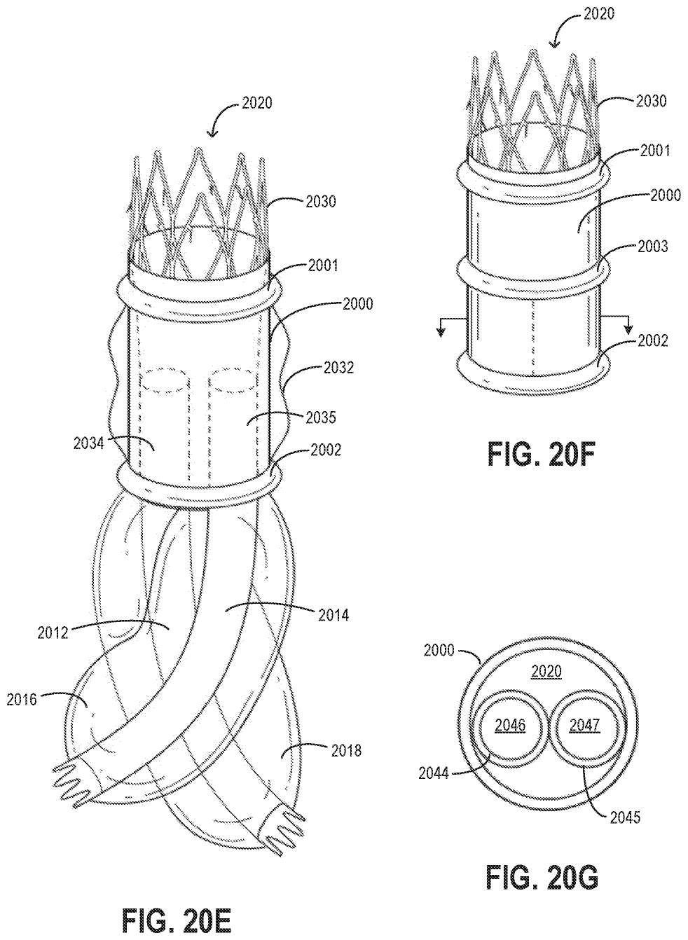

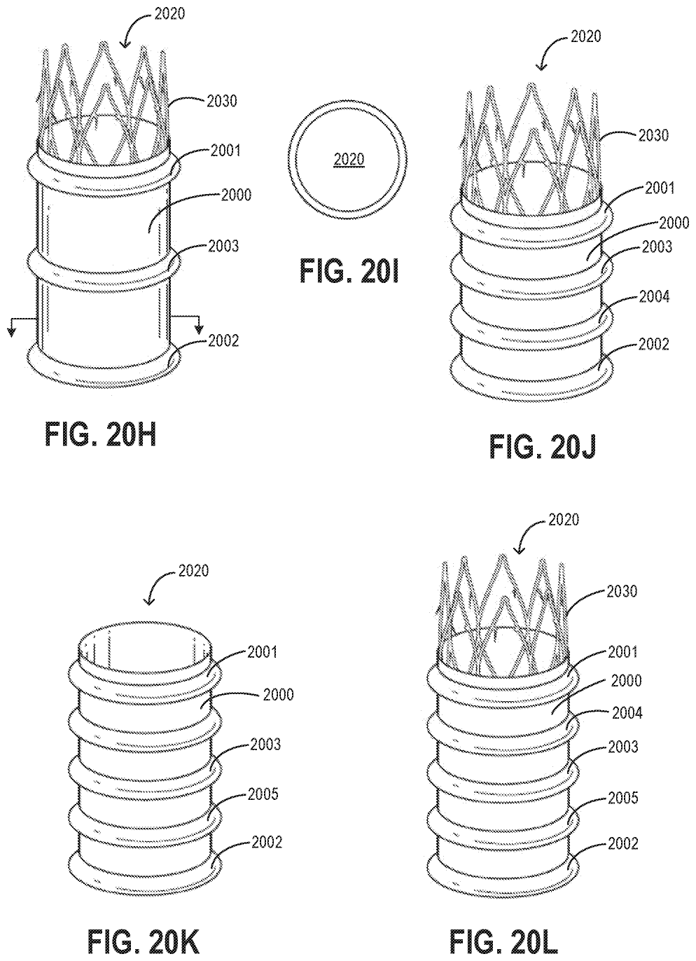

FIGS. 20A, 20B, 20C, 20D, 20E, 20F, 20G, 20H, 20I, 20J, 20K, and 20L illustrate examples of a proximal graft 2000 according to various arrangements. Referring to FIGS. 1, 20A, 20B, 20C, 20D, 20E, 20F, 20G, 20H, 20I, 20J, 20K, and 20L, the proximal graft 2000 is a graft component made of graft material. The proximal graft 2000 has a proximal end, a distal end, an internal surface, and an external surface. The proximal end of the proximal graft 2000 is the end of the proximal graft 2000 that is closer to or in the proximal neck region 17 when deployed. The distal end of the proximal graft 2000 is the end of the proximal graft 2000 that is closer to the aortic bifurcation 11 when deployed. Typically, the distal end of the proximal graft 2000 can be placed into the sac of the aneurysm 14. The proximal graft 2000 has a cylindrical shape and forms a bore or tubular lumen 2020. The internal surface of the proximal graft 2000 faces the tubular lumen 2020. The external surface of the proximal graft 2000 faces walls/surfaces of the aorta 10 when deployed and faces away from the lumen 2020 of the proximal graft 2000. Blood is configured to flow through the lumen 2020.

The proximal graft 2000 includes at least one support component. Each support component can be embedded in the proximal graft 2000. In some arrangements, the support component is a support inflatable fill structure surrounding the proximal graft 2000 such as but not limited to, support rings or balloons made from a polymer (e.g., PTFE, polyurethane, and so on). In some arrangements, the support component is attached, fixed, bonded (e.g., thermally bonded), sutured, or otherwise coupled to the proximal graft 2000 such that an interior portion (including an inner surface portion) of each support component is inside of the lumen 2020 while the remaining exterior portion (including an outer surface portion) of the support component is outside of the proximal graft 2000 and is coupled to the external surface of the proximal graft 2000. In other arrangements, the support component is attached, fixed, bonded (e.g., thermally bonded), sutured, or otherwise coupled to the external surface of the proximal graft 2000. The support component can be inflated using a suitable fill line.

In FIG. 20A, the proximal graft 2000 includes two support components 2001 and 2002. The support component 2001 is located at the proximal end of the proximal graft 2000 while the support component 2002 is located at the proximal end of the proximal graft 2000.

In FIG. 20B, the proximal graft 2000 further includes an anchor 2030. The anchor 2030 is a fixation feature, a fixation stent frame, and so on. The anchor 2030 anchors, fixes, or attaches the proximal end of the proximal graft 2000 to the walls/surfaces of the aorta 10, in the manner described with respect to the anchor 245.

In FIG. 20C, the proximal graft 2000 further includes an inflatable structure 2032. The inflatable structure 2032, in the inflated state, can expand radially toward the surfaces/walls of the aorta 10 to fill one or more of the sac of the aneurysm 14 (for sack management), a space between the external surface of the proximal graft 2000 and the surfaces/walls of the proximal neck region 17 (for neck sealing), and a space between the external surface of limb stent grafts (e.g., limb stent grafts 2012 and 2014) and the surfaces/walls of the iliac arteries 12 and 13, in the manner described herein. The inflatable structure 2032 can be attached, fixed, bonded (e.g., thermally bonded), sutured, or otherwise coupled to at least a portion of the external surface of the proximal graft 2000.

In FIGS. 20D and 20E, the proximal graft 2000 includes the support components 2001 and 2002, the anchor 2030, the inflatable structure 2032, and a bifurcation feature including lumens 2034 and 2035. That is, the proximal graft 2000 is shaped such that the lumen 2020 that is located at the proximal end of the proximal graft 2000 becomes bifurcated into the lumens 2034 and 2035 in a docking zone at the distal end of the proximal graft 2000. The proximal end and the distal end are opposite ends of the proximal graft 2000. The limb stent grafts 2012 and 2014 can be docked or inserted into the lumens 2034 and 2035 in the manner described herein. The limb stent grafts 2012 and 2014 include respective ones of the inflatable structures 2016 and 2018 for sac management and sealing in the manner described herein.

In FIGS. 20F and 20G, the proximal graft 2000 includes the support components 2001-2003 and the anchor 2030. The support component 2003 is between the support components 2001 and 2002 along the proximal graft 2000. The support components 2001-2003 are spaced apart from each other along the proximal graft 2000. In some arrangements, the proximal graft 2000 includes inner sleeves or rings 2044 and 2045 that form lumens 2046 and 2047, respectively, for receiving the limb stent grafts 2012 and 2014. The inner sleeves or rings 2044 and 2045 are within the lumen 2020 in a docking zone between the support components 2002 and 2003. In some examples, the inner sleeves or rings 2044 and 2045 can be sleeves or support rings made from a polymer (e.g., PTFE, polyurethane, and so on). The inner sleeves or rings 2044 and 2045 and the bifurcation feature can eliminate leakage from the gutters.

In FIGS. 20H and 20I, the proximal graft 2000 includes the support components 2001-2003 and the anchor 2030, and without any bifurcation features or inner sleeves. In FIG. 20J, the proximal graft 2000 includes the support components 2001-2004 and the anchor 2030. The support components 2003 and 2004 are between the support components 2001 and 2002 along the proximal graft 2000. The support components 2001-2004 are spaced apart from each other along the proximal graft 2000.

In FIG. 20K, the proximal graft 2000 includes the support components 2001-2005. The support components 2003-2005 are between the support components 2001 and 2002 along the proximal graft 2000. The support components 2001-2005 are spaced apart from each other along the proximal graft 2000. In FIG. 20L, the proximal graft 2000 includes the support components 2001-2005 and the anchor 2030.

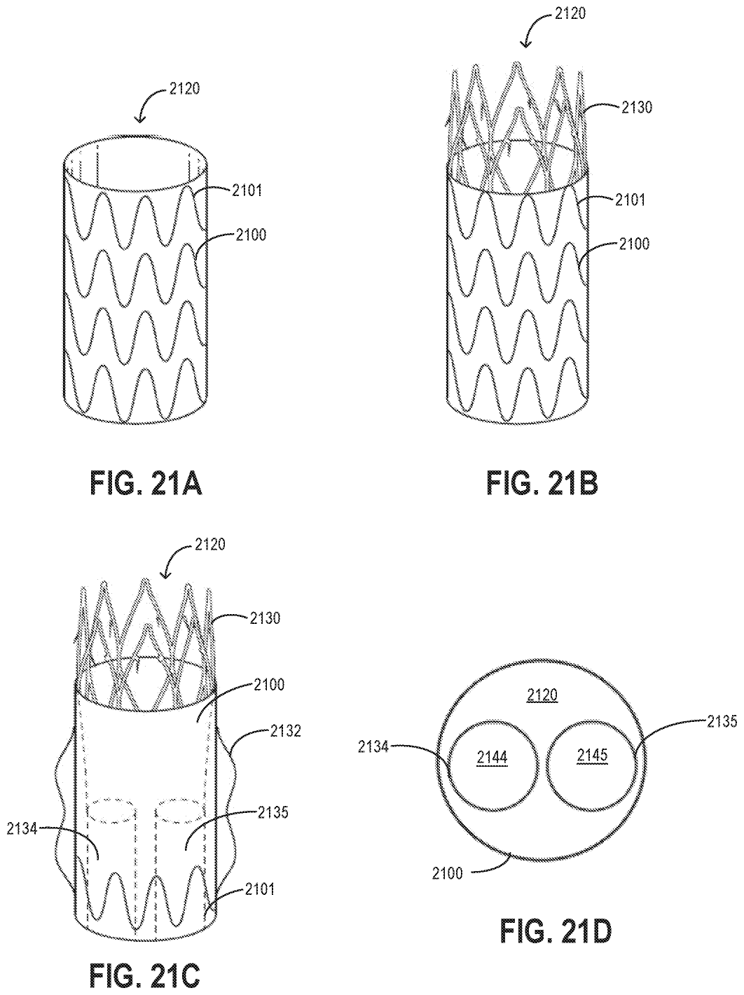

FIGS. 21A, 21B, 21C, and 21D illustrate examples of a proximal extension stent graft 2100 according to various arrangements. Referring to FIGS. 1, 21A, 21B, 21C, and 21D, the proximal extension stent graft 2100 is a proximal stent graft. The proximal extension stent graft 2100 has a proximal end, a distal end, an internal surface, and an external surface. The proximal end of the proximal extension stent graft 2100 is the end of the proximal extension stent graft 2100 that is closer to or in the proximal neck region 17 when deployed. The distal end of the proximal extension stent graft 2100 is the end of the proximal extension stent graft 2100 that is closer to the aortic bifurcation 11 when deployed. Typically, the distal end of the proximal extension stent graft 2100 can be placed into the sac of the aneurysm 14. The proximal extension stent graft 2100 has a cylindrical shape and forms a bore or tubular lumen 2120. The internal surface of the proximal extension stent graft 2100 faces the lumen 2120. The external surface of the proximal extension stent graft 2100 faces walls/surfaces of the aorta 10 when deployed and faces away from the lumen 2120. Blood is configured to flow through the lumen 2120. The proximal extension stent graft 2100 includes wire-wound stents 2101 that has multiple wire-wound rings.

FIG. 21B shows the proximal extension stent graft 2100 further including an anchor 2130 that is similar to the anchor 2030. FIG. 21C shows the proximal extension stent graft 2100 further including an inflatable structure 2132 similar to the inflatable structure 2032.

The proximal extension stent graft 2100 shown in FIGS. 21C and 21D includes encapsulated wire-wound stents 2134 and 2135 in the docking zone on the distal end of the proximal extension stent graft 2100. The encapsulated wire-wound stents 2134 and 2135 form lumens 2144 and 2145 within the lumen 2120. Such encapsulated wire-wound stents 2134 and 2135 allow limb stent grafts to dock in the lumen 2120 of the proximal extension stent graft 2100. The distal ends of the limb stent grafts are oversized (e.g., having a diameter larger than the diameter of the lumens 2144 and 2145), such that the limb stent grafts create outward radial force relative to the encapsulated wire-wound stents 2134 and 2135 to ensure that the limb stent grafts and the encapsulated wire-wound stents 2134 and 2135 remain joined.

In some arrangements, the grafts 2000 and 2100 is a straight rigid bore. In some arrangements, the grafts 2000 and 2100 is made from a more flexible PTFE material. In the arrangements in which the grafts 2000 and 2100 is made from the flexible PTFE material, the grafts 2000 and 2100 can function like an active seal as blood pressure inside of the lumens 2020 and 2120 pushes the walls of the grafts 2000 and 2100 against vessel walls of the aorta 10. Thus, the active seal is formed between the external surfaces of the grafts 2000 and 2100 and the vessel walls of the aorta 10.

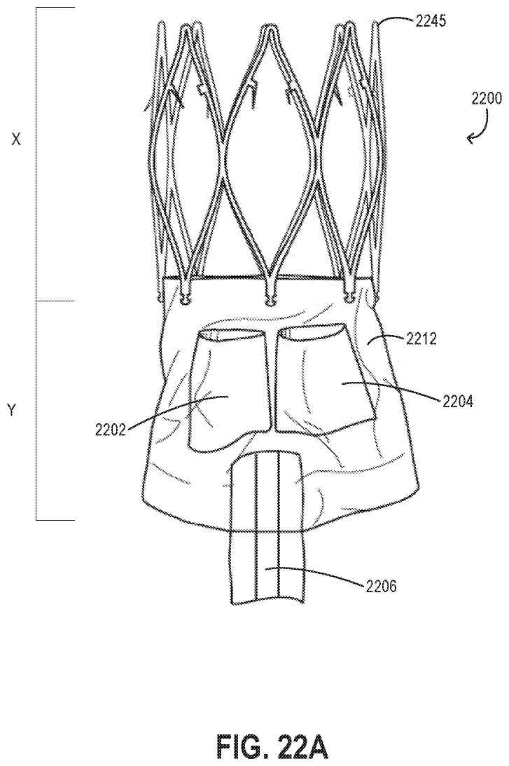

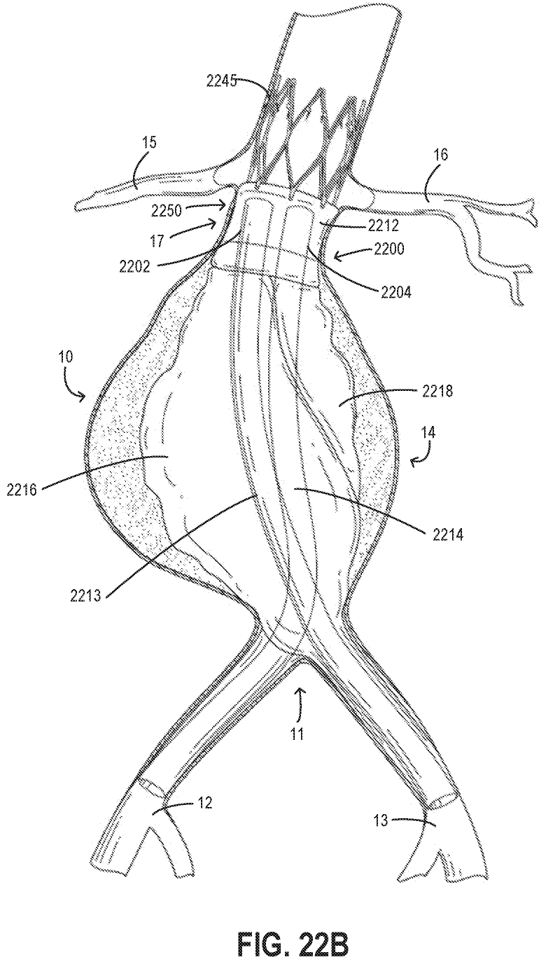

FIG. 22A illustrates an example proximal extension inflatable fill structure 2212 of a system 2200 according to various arrangements. FIG. 22B is a cross-sectional view of the system 2200 (FIG. 22A) deployed across the aneurysm 14 (FIG. 1) according to various arrangements. Referring to FIGS. 1, 22A, and 22B, the system 2200 includes the proximal extension inflatable fill structure 2212, a first limb stent graft 2213, a second limb stent graft 2214, an anchor 2245, an inflatable fill structure 2216, and an inflatable fill structure 2218.

In some example, the proximal extension inflatable fill structure 2212 is an inflatable fill structure (e.g., an endobag). In various examples, the proximal extension inflatable fill structure 2212 has a wider polymer-filled seal zone compared to a seal ring on other devices. The width of the proximal extension inflatable fill structure 2212 is denoted as Y as shown. In some examples, Y is approximately 20 mm. As discussed herein, the wider proximal extension inflatable fill structure 2212 is forgiving in placement accuracy, even if the proximal extension stent graft 2000 is placed lower (e.g., 1 mm lower) than an optimal position, the wider proximal extension inflatable fill structure 2212 can nevertheless provide a tight seal in the proximal neck region 17. The wider proximal extension inflatable fill structure 2212 also has a wider treatment diameter range, which means fewer number of sizes (and fewer number of SKUs) are needed for treating the entire vessel treatment range. In some arrangements, a neck length of the proximal extension inflatable fill structure 2212 is shorter than the neck lengths of other devices. Furthermore, the wide proximal extension inflatable fill structure 2212 can improve the neck angle indication.

In some arrangements the proximal extension inflatable fill structure 2212 has or is in communication with a fill line 2206 through which hardenable inflation materials or fill polymers (e.g., polyesters, PTFE, polyurethane, and the like) are communicated in liquid form. The proximal extension inflatable fill structure 2212 can be deployed in the proximal neck region 17 and inflated therein using the fill line 2206. The proximal extension inflatable fill structure 2212, in the inflated state, forms a seal in the proximal neck region 17 to eliminate Type II Endoleaks. The proximal extension inflatable fill structure 2212 can be filled to a higher pressure than other devices. The proximal extension inflatable fill structure 2212 can also provide a more accurate seal zone and a more circumferential seal in the proximal neck region 17. The proximal extension inflatable fill structure 2212 can prevent the inflatable fill structures 2216 and 2218 from prolapsing into the renal arteries 15 and 16 when the inflatable fill structures 2216 and 2218 are being inflated or when the limb stent grafts 2213 and 2214 are docking in a docking zone 2250. The lumens 2202 and 2204 are also located in the proximal neck region 17 when the proximal extension inflatable fill structure 2212 forms the seal in the proximal neck region 17.

As shown, the proximal extension inflatable fill structure 2212 from lumens 2202 and 2204 to which the limb stent grafts 2213 and 2214 are docked. The lumens 2202 and 2204 correspond to the docking zone 2250. When the proximal extension inflatable fill structure 2212 is in the inflated state, the lumens 2202 and 2204 are fully expanded. The sizes of the fully expanded lumens 2202 and 2204 are slightly smaller than the sizes of the proximal ends of the limb stent grafts 2213 and 2214. Given the elasticity of the material of the proximal extension inflatable fill structure 2212 (in the inflated state), the material of the proximal extension inflatable fill structure 2212 around the lumens 2202 and 2204 forms a seal as the limb stent grafts 2213 and 2214 are docked therein.

In various arrangements, the anchor 2245 (a fixation feature, a fixation stent frame, and so on) anchors, fixes, or attaches the proximal end of the proximal extension inflatable fill structure 2212 to the walls/surfaces of the aorta 10, prevents intrusion of blood into a region between an outer wall and an inner surface of the aneurysm 14, and improves the transition from the aorta 10 into the lumens of the proximal extension inflatable fill structure 2212. In some examples, the anchor 2245 is stitched or sutured onto the proximal extension inflatable fill structure 2212. In some examples, the anchor 2245 can include a stent, graft, and/or other expandable luminal support structure. In some examples, the anchor 2245 is self-expanding and includes a suprarenal laser-cut stent with coils attached thereon. In some examples, the anchor 2245 has a stent shorter than that of some current stent graft systems to eliminate free crowns. The length of the anchor 2245 is denoted as X. In some examples, X is approximately 30 mm or less. A shorter stent allows for a larger neck angle indication due to an improved stent graft flexibility. As such, the suprarenal stent of the anchor 2245 is shorter and has fewer crowns and fewer anchors, allowing the systems 2200 to be used for smaller treatment sizes. That is, the stent graft system 2200 is a low-profile delivery system that can be used for small treatment sizes.

The inflatable fill structure 2216 is fixed, bonded, attached, or otherwise coupled to at least a portion of the outer surface of the limb stent graft 2213. The inflatable fill structure 2218 is fixed, bonded, attached, or otherwise coupled to at least a portion of the outer surface of the limb stent graft 2214. Each of the inflatable fill structures 2216 and 2218 can be inflated using a dedicated fill line or a fill line shared with another component of the system 2200. When inflated, the inflatable fill structures 2216 and 2218 expand radially from the limb stent grafts 2213 and 2214 toward surfaces/walls of the sac of the aneurysm 14. In the inflated state, the inflatable fill structures 2216 and 2218 surround the limb stent grafts 2213 and 2214, respectively.

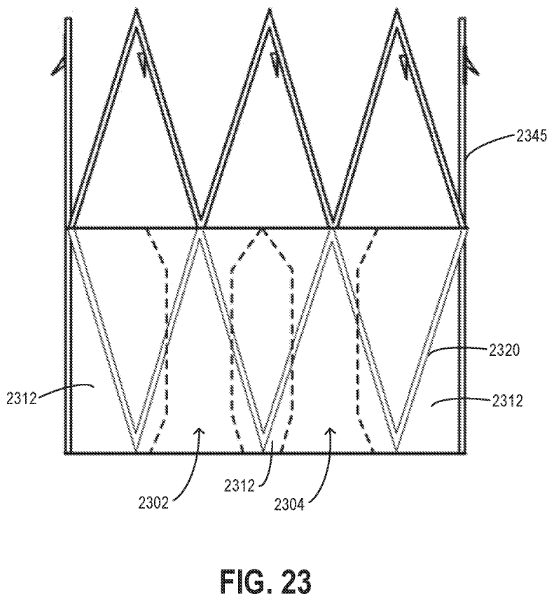

FIG. 23 illustrates an example proximal extension inflatable structure of a stent graft system according to various arrangements. Referring to FIGS. 1 and 23, an anchor 2245 (a fixation feature, a fixation stent frame, and so on) anchors, fixes, or attaches a proximal end of the proximal extension inflatable fill structure 2312 to the walls/surfaces of the aorta 10. The proximal extension inflatable fill structure 2312 can be an element such as but not limited to, the proximal extension inflatable fill structure 2212. In some examples, the anchor 2345 is stitched or sutured onto the proximal extension inflatable fill structure 2312. In some examples, the anchor 2345 can include a stent, graft, and/or other expandable luminal support structure. In some examples, the anchor 2345 includes stents that are connected to or extends from the stents 2320 of the proximal extension inflatable fill structure 2312. As shown, the anchor 2345 includes hooks or barbs for fixation. In some examples, the anchor 2345 is self-expanding and includes a suprarenal laser-cut stent with coils attached thereon. The proximal extension inflatable fill structure 2312, when inflated, can form two lumens 2302 and 2304 similar to the lumens 2202 and 2204.

Accordingly, in some arrangements, the stent graft system described herein includes a wider seal ring that improves placement accuracy while providing a wider treatment diameter range. In some arrangements, inflatable fill structures (e.g., endobags) can be provided to prevent Type II Endoleaks. In some arrangements, a proximal graft having a large bore diameter is easier to cannulate than the much smaller contra lumen in some other devices. The proximal graft having the large bore diameter can also reduce or eliminates the possibility of cannulating the wrong side (ipsi) lumen.

The present technology is not to be limited in terms of the particular arrangements described in this application, which are intended as illustrations of aspects of the present technology. Many modifications and variations of this present technology can be made without departing from its spirit and scope, as will be apparent to those skilled in the art. Functionally equivalent systems and methods within the scope of the present technology, in addition to those enumerated herein, will be apparent to those skilled in the art from the foregoing descriptions. Such modifications and variations are intended to fall within the scope of the present technology. It is to be understood that this present technology is not limited to particular systems and methods of using systems, which can, of course, vary. It is also to be understood that the terminology used herein is for the purpose of describing particular arrangements only and is not intended to be limiting.

1. A stent graft system, comprising:
a first graft;
a second graft; and
a third graft, wherein
each of the first graft, the second graft, and the third graft are separate grafts before being deployed and each comprises at least one lumen;
wherein the second graft and the third graft are configured to be inserted into the lumen of the first graft when deployed.

2.-4. (canceled)

5. The stent graft system of claim 1, further comprising:
a seal component coupled to the first graft, the seal component forms a seal in a proximal neck region of the aorta.

6. The stent graft system of claim 5, wherein the system further comprises an inflatable fill structure at least partially surrounding the first graft, the fill structure being configured to expand within and contact the aorta wall.

7. The stent graft system of claim 5, further comprising an anchor coupled to the first graft.

8. The stent graft system of claim 5, wherein the inflatable fill structure, when deployed, at least partially surrounds proximal ends of the second graft and the third graft that are docked within the single lumen of the first graft.

9. The stent graft system of claim 5, wherein
the inflatable fill structure is coupled to the first graft;
the second graft and the third graft dock within the single lumen of the first graft in a docking zone; and
the inflatable fill structure, in an inflated state, surrounds at least portions of the second graft and the third graft that are outside of the docking zone.

10.-12. (canceled)

13. The stent graft system of claim 5, wherein
the second graft and the third graft dock within the single lumen of the first graft in a docking zone; and
the first graft comprises a wire-wound stent component coupled to a portion of the first graft in the docking zone, the wire-wound stent component comprises a plurality of wire-wound rings.

14. (canceled)

15. The stent graft system of claim 5, wherein
the second graft and the third graft dock within the single lumen of the first graft in a docking zone;
the first graft comprises a wire-wound stent ring coupled to a portion of the first graft in the docking zone, the wire-wound stent ring comprises a single ring of wire-wound stent.

16. (canceled)

17. The stent graft system of claim 5, wherein the inflatable fill structure forms a funnel shape in an inflated state.

18. (canceled)

19. The stent graft system of claim 5, wherein the inflatable fill structure is a bifurcated inflatable fill structure that, when in an inflated state, forms two lumens for receiving the second graft and the third graft.

20. The stent graft system of claim 1, further comprises
a first inflatable fill structure at least partially surrounding the first graft;
a second inflatable fill structure at least partially surrounding the second graft; and
a third inflatable fill structure at least partially surrounding the third graft, the first inflatable fill structure, the second inflatable fill structure, and the third inflatable fill structure are separate inflatable fill structures that expand within the aorta when deployed.

21.-24. (canceled)

25. The stent graft system of claim 1, further comprising:
a first inflatable fill structure at least partially surrounding the second graft; and
a second inflatable fill structure at least partially surrounding the third graft, wherein the first inflatable fill structure and the second inflatable fill structure expand within the aorta and surrounds at least partially the first graft when deployed.

26. The stent graft system of claim 25, wherein each of the second graft and the third graft comprises a wire-wound stent component, the wire-wound stent component comprises a plurality of wire-wound rings.

27.-34. (canceled)

35. The stent graft system of claim 1, wherein the first graft comprises a laminated stent component.

36.-40. (canceled)

41. A stent graft system, comprising:
a graft forming a lumen;
at least one support component embedded in the graft, each of the at least one support component is a polymer ring surrounding the graft, at least a portion of each of the at least one support component is coupled to an external surface of the graft, the external surface faces away from the lumen, wherein
the at least one support component comprises a first support component and a second support component;
the first support component is located on a first end of the graft; and the second support component is located on a second end of the graft.

42.-44. (canceled)

45. The stent graft system of claim 41, wherein the graft further comprises inner sleeves or rings in the lumen that receive limb grafts.

46. A system, comprising:
a proximal extension inflatable fill structure, configured to form a seal in a proximal neck region of an aorta when the proximal extension inflatable fill structure is inflated; and
configured to form at least one lumen when the proximal extension inflatable fill structure is inflated, wherein in one or more of the at least one lumen is configured to receive a limb stent graft; and
a second graft; and
a third graft,
wherein each of the first graft, the second graft, and the third graft are separate grafts before being deployed forms and each comprises at least one a single lumen;
wherein the second graft and the third graft are configured to be inserted into the lumen of the inflatable fill structure when deployed, and
when deployed, the first graft, the second graft, and the third graft are coupled together.

47. The system of claim 46, further comprising an anchor coupled to the proximal extension inflatable fill structure.

48. The system of claim 46, wherein the proximal extension inflatable fill structure forms a dual lumen.

49. The system of claim 46, wherein the second and third grafts are placed in separate lumen in the proximal extension inflatable fill structure.

50. (canceled)
